(19) United States
(12) Patent Application Publication
(10) Pub. No.: US 2026/0366036 A1
(43) Pub. Date: Nov. 19, 2026

(54) GENETICALLY MODIFIED SOYBEAN WITH REDUCED TRYPSIN AND CHYMOTRYPSIN INHIBITOR ACTIVITY

(71) Applicants: The Curators of the University of Missouri, Columbia, MO (US); The United States of America, as Represented by the Secretary of Agriculture, Washington, DC (US)

(72) Inventors: Hari B. Krishnan, COLUMBIA, MO (US); Won-Seok Kim, COLUMBIA, MO (US); Jason D. Gillman, COLUMBIA, MO (US)

(73) Assignees: The Curators of the University of Missouri, Columbia, MO (US); The United States of America, as Represented by the Secretary of Agriculture, Washington, DC (US)

(21) Appl. No.: 19/675,630

(22) Filed: May 13, 2026

Related U.S. Application Data
(60) Provisional application No. 63/804,955, filed on May 13, 2025.

(51) Int. Cl. C12N 15/82; A23K 10/30

(52) U.S. Cl. CPC C12N 15/8242; A23K 10/30

(57) ABSTRACT

Provided are genetically modified soybean seeds, soybean plants and parts thereof, as well as related plant products including soybeans and soybean meal, and animal feeds and human food products comprising same, and methods for producing same.

### FIELD OF THE INVENTION

The present invention relates to genetically modified soybean seeds, plants and related products and methods for producing soybeans having low levels of trypsin and chymotrypsin inhibitor activity.

### STATEMENT CONCERNING BIOLOGICAL DEPOSIT

A deposit is being made with the American Type Culture Collection (ATCC), 10801 University Boulevard, Manassas, Virginia 20110-2209, in connection with this application. During the pendency of the subject application, access to the deposit shall be afforded to the Commissioner upon request. To the extent required by law, all restrictions upon public access to this deposit shall be removed upon the grant of a patent on this application and the deposits shall be replaced if viable samples cannot be made by the depository named hereinabove.

### BACKGROUND

Soybean Glycine max (L.) Merr is grown worldwide as a rotation partner for grain crops, particularly maize, due to an ability to fix atmospheric nitrogen, high seed yield potential, relatively few major biotic pathogens, and relatively low input costs. Soybean seeds contain far more protein than other legumes (on average 40%), and soybean meal is often the first choice for swine and poultry feed mixtures due to a balanced (albeit suboptimal) amino acid profile. Soybean meal, produced after extraction of seed oil, has a relatively low cost and is of high quality compared to other competing protein meal sources.

Despite the high quality of soybean protein, raw soybeans and soybean meal cannot be directly included in animal feed mixtures due to anti-nutritional compounds that interfere with digestion, cause pancreatic inflammation, and effectively reduce animal weight gain. Soybean seeds contain two major classes of proteinaceous anti-nutritional factors, which were named after the scientists that discovered them: (1) Kunitz trypsin inhibitors (KTis), which inactivate the digestive enzyme trypsin, and (2) Bowman-Birk inhibitors (BBis), which inactivate both trypsin and chymotrypsin animal digestive enzymes. In soybean, three KTi isoforms (KTi1, KTi2, and KTi3) have been reported. There are at least 11 different BBi-encoding genes present in the soybean genome. However, expression analysis of RNAseq data reveals that only three of them are expressed at appreciable levels in developing seeds.

Experiments have conclusively shown that ingestion of high levels of active protease inhibitors is responsible for reduced animal weight gain and can even result in hypoglycemia, pancreatic hypertrophy, or liver damage. To avoid these issues and permit the inclusion of soybean meal at high levels in animal feed, KTi and BBi anti-nutritional proteins in typical soybean seeds must be inactivated by heat treatment, which has been shown to reduce, but not eliminate, ANF activity. Heat inactivation allows soybean meal to be included at moderately high levels in chicken and swine feed mixtures. While this step meets the needs of the industry, such treatment is energy-intensive, adds expense, and requires a balance between sufficiently reducing ANF activity and negatively impacting the quality of seed proteins.

Heat treatment could possibly be reduced, or eliminated, via the identification or production of soybeans having reduced trypsin and chymotrypsin inhibitor activity. One success story in genetically reducing soybean's trypsin inhibitor activity was the identification and analysis of a soybean accession (PI 157740), which features a marked reduction (~40%) in trypsin inhibitor activity. This genetic source has been examined through both genetic analysis and animal feeding trials. Raw extruded protein meal lacking KTi3 protein was found to result in superior animal weight gain compared with raw soybeans but not to a degree that could eliminate the need for heat inactivation. Accordingly, other options are needed to address the problems associated with heat treatment and the presence of anti-nutritional factors in soybeans.

### BRIEF SUMMARY

The present invention provides genetically modified soybeans, soybean seeds, and related products, including soybean meal and flour, having reduced trypsin and chymotrypsin inhibitor activity, as well as related products including animal feed and food for human consumption having higher protein digestibility and higher amino acid adsorption, such as tofu, soy milk, plant based meat and fermented foods. Also provided are methods for making the genetically modified soybean seeds and plants described herein.

Accordingly, in one aspect, provided is a genetically modified soybean seed comprising a loss of function mutation in at least one Bowman-Birk protease inhibitor (BBi) gene, where the soybean seed has at least a 2-fold reduction in chymotrypsin inhibitor activity and at least a 2-fold reduction in trypsin inhibitor activity compared to a reference soybean seed without the BBi mutation. The genetically modified soybean seed may also include where the loss of function mutation results in a reduction or elimination of expression of at least one BBi gene compared to a reference soybean seed without the mutation. The genetically modified soybean seed may also include where the at least one BBi gene is Glyma.16g208900 or Glyma.14G117700, or both. The genetically modified soybean seed may also include where the seed contains similar amounts of Kunitz protease inhibitor (KTi) protein compared to a reference soybean seed without the BBi mutation. In specific embodiments, the genetically modified soybean seed comprising a loss of function mutation in at least one BBi gene is designated USDA-168-1 (ZB168-1), USDA-168-4 (ZB168-4), USDA-168-5 (ZB168-5), USDA-Cas9-2.8 (Event 2), USDA-Cas9-7.2 (Event 7) or USDA-Cas9-8.7 (Event 8). The designations in parenthesis are alternative names for the same seed, and are utilized in the examples infra. In some aspects, the genetically modified soybean seed may also comprise a loss of function mutation in one or more Kunitz protease inhibitor (KTi) isoforms, in addition to the loss of function mutation in the at least one BBi gene. In a specific embodiment, the genetically modified soybean seed comprising a loss of function mutation in at least one BBi gene and at least one KTi gene is designated USDA-HBK-G7.8 (G78). The genetically modified soybean seed may also include where the loss of function mutation in the at least one BBi gene is a deletion, insertion, or frameshift mutation resulting in one or more of reduced BBi gene expression, an enzymatically inactive BBi protein or a BBi protein having reduced enzymatic activity compared to a reference BBi protein without the mutation. The genetically modified soybean seed may also include where the species of the soybean seed is Glycine max (L.) Merrill. The genetically modified soybean seed may also include where the at least one BBI gene comprises one or more of Glyma.09G158500, Glyma.09G158600, and Glyma.09G158700. In some aspects, the genetically modified soybean seed may comprise a loss of function mutation in a KTi1, KTi2, or KTi3 isoform, or in both KTi1 and KTi3 isoforms.

Also provided is a genetically modified soybean or soybean meal comprising a loss of function mutation in at least one Bowman-Birk protease inhibitor (BBi) gene, where the soybean or soybean meal has at least a 2-fold reduction in chymotrypsin inhibitor activity and at least a 2-fold reduction in trypsin inhibitor activity compared to a reference soybean or soybean meal without the BBi mutation. In aspects, the at least one BBi gene is Glyma.16g208900 or Glyma.14G117700, or both. Preferably, the genetically modified soybean or soybean meal has not been subjected to heat treatment, or if heat treatment is necessary, the heat treatment is at a lower temperature or for a reduced period of time, or both, compared to what is required to produce a similar level of residual trypsin inhibitor activity in soybean or soybean meal not carrying a BBi mutation, or a combination of a BBi mutation and a KTi mutation, as described herein. The genetically modified soybean or soybean meal may also include where trypsin or chymotrypsin inhibitor activity measured in the soybean or soybean meal is less than 50%, less than 20% or less than 10% of the activity of a reference soybean or meal not carrying a BBi mutation, or a combination of a BBi mutation and a KTi mutation, as described herein. The genetically modified soybean or soybean meal may also include where the trypsin inhibitor activity is less than 2.5 milligrams/gram or less than 0.25% by weight. The genetically modified soybean or soybean meal may also include where the species of the soybean is Glycine max (L.) Merrill. The genetically modified soybean or soybean meal may also include where the soybean is a Maverick cultivar. Also provided is an animal feed mixture comprising a genetically modified soybean or soybean meal as described herein.

Also provided is a method for reducing trypsin and chymotrypsin inhibitor activity in soybean or soybean seed, or in a meal or flour produced from the soybean or soybean seed, the method comprising producing a transgenic soybean plant comprising one or more loss of function mutations in at least one Bowman-Birk protease inhibitor (BBi) gene. The method may also include where the at least one BBi gene includes one or both of Glyma.16g208900 and Glyma.14G117700. The method may also include where trypsin or chymotrypsin inhibitor activity of the soybean, seed, or a meal or flour produced from the soybean seed, is less than 50% or less than 20% or less than 10% of the activity of a reference soybean or seed not carrying the BBi mutation, or a meal or flour produced from the reference soybean seed. The method may also include where the trypsin inhibitor activity of the soybean or soybean seed, or in a meal or flour produced from the soybean seed, is less than 2.5 milligrams/gram or less than 0.25%. Preferably, the method does not comprise subjecting the soybean or seed, or a meal or flour produced from the soybean seed, to a heat treatment. However, where a heat treatment is required or desirable, the heat treatment is preferably a modified heat treatment which may include one or more of exposure to a lower temperature and/or exposure to the temperature for a shorter period of time compared to a heat treatment required to produce the same trypsin or chymotrypsin inhibitor activity in a soybean, seed, or a meal or flour produced from the soybean seed not carrying a BBi mutation as described herein. Standard heat treatment processes to which the claimed modified heat treatment may be compared typically include one or more of roasting, extrusion, and/or steam treatment where the heat treatment includes exposure to a temperature of between 100-110 °C for a period of time ranging from minutes to hours, optionally followed by exposure to a temperature up to 150 °C.

In aspects, the method does not comprise mutating a Kunitz protease inhibitor (KTi) gene. In other aspects, the soybean includes a loss of function mutation in one or more Kunitz protease inhibitor (KTi) gene isoforms, optionally where the KTi isoform is a KTi1, KTi2, or KTi3 isoform. In aspects, the one or more loss of function mutations in the at least one BBi gene is introduced by a method comprising Agrobacterium-mediated transformation of an expression cassette encoding at least one guide RNA targeting the at least one BBi gene. The method may also include where the one or more loss of function mutations is introduced using a CRISPR/Cas9 system. The method may also include where the species of the transgenic soybean plant is Glycine max (L.) Merrill. The method may also include where the transgenic soybean plant is a Maverick cultivar.

In another aspect, provided is a genetically modified soybean cell line comprising a loss of function mutation in at least one Bowman-Birk protease inhibitor (BBi) gene, where cells of the cell line have at least a 2-fold reduction in chymotrypsin inhibitor activity and at least a 2-fold reduction in trypsin inhibitor activity compared to a reference soybean cell without the BBi mutation. The genetically modified soybean cell may also include where the at least one BBi gene comprises one or more of Glyma.16G208900, Glyma.14G117700 and Glyma.09G158700. The genetically modified soybean cell may also include where the cell line comprises a loss of function mutation in one or more Kunitz protease inhibitor (KTi) gene isoforms, optionally where the KTi isoform is a KTi1, KTi2, or KTi3 isoform. The genetically modified soybean cell line may also include where cells of the cell line do not express BBi or KTi proteins. The genetically modified soybean cell line may also include where cells of the cell line are negative for chymotrypsin and trypsin inhibitor activities as measured in an in vitro assay utilizing N-Succinyl-Ala-Ala-Pro-Phe p-nitroanilide and N-benzoyl-D,L-arginine 4-nitroanilide as substrates, respectively.

Also provided is a soybean plant, a part thereof, or a cell thereof produced by growing a genetically modified soybean seed as described herein. In specific embodiments, the genetically modified soybean seed carries an inactivating mutation in one or more BBi genes and is designated USDA-168-1 (ZB168-1), USDA-168-4 (ZB168-4), USDA-168-5 (ZB168-5), USDA-Cas9-2.8 (Event 2), USDA-Cas9-7.2 (Event 7), USDA-Cas9-8.7 (Event 8), or USDA-HBK-G7.8 (G78). In aspects, the soybean plant or the part thereof is selected from the group consisting of a leaf, a flower, a soybean, a stalk, a root, a rootstock, a scion, a peduncle, a stamen, a pistil, and a meristem. Also provided is a line of regenerable cells produced from the soybean plant or the part thereof, wherein the soybean plant regenerated from a tissue culture of the cells has all of the physiological and morphological characteristics of the genetically modified soybean seed designated USDA-168-1 (ZB168-1), USDA-168-4 (ZB168-4), USDA-168-5 (ZB168-5), USDA-Cas9-2.8 (Event 2), USDA-Cas9-7.2 (Event 7), or USDA-Cas9-8.7 (Event 8) or USDA-HBK-G7.8 (G78). Also provided are progeny of a genetically modified soybean seed as described herein produced from the cross of the plant or plant part of the genetically modified soybean seed with a different Glycine max (L.) Merrill plant. In one aspect, such progeny is designated USDA-HBK-G7.8 (G78).

Other technical features may be readily apparent to one skilled in the art from the following figures, descriptions, and claims.

### BRIEF DESCRIPTION OF THE DRAWINGS

FIG. 1A is a schematic illustration showing a summary of CRISPR-Cas9 mutations identified in Glyma.16g208900, the most highly expressed BBi isoform, in six independent events.

FIG. 1B is a schematic diagram showing the effect of CRISPR-CAS9-induced deletions, insertions, and frameshift mutations on the amino acid sequence of Glyma.16g208900. *=stop codon.

FIG. 2A is a schematic diagram showing a summary of the CRISPR-Cas9 mutations identified in Glyma.14G117700, the second-most highly expressed BBi isoform, in six independent events.

FIG. 2B is a schematic diagram showing the effect of CRISPR-CAS9-induced deletions, insertions, and makeshift mutations on the amino acid sequence of Glyma.14G117700. *=stop codon.

FIG. 3A is an image showing the proteomic analysis of total seed proteins from CRISPR-Cas9 BBi mutant and control lines using sodium dodecyl sulfate polyacrylamide gel electrophoresis visualized with Coomassie Blue. Samples in order are ‘Maverick’ (Mav., non-transgenic control); 2=Event 2; 3=Event 3; 4=Event 4; 5=Event 5; 7=Event 7; 8=Event 8.

FIG. 3B is an image of an immunoblot analysis of CRISPR-Cas9 BBi mutant and control lines using β-conglycinin-specific antibodies. Samples in order are ‘Maverick’ (Mav., non-transgenic control); 2=Event 2; 3=Event 3; 4=Event 4; 5=Event 5; 7=Event 7; 8=Event 8.

FIG. 3C is an image of an immunoblot analysis of CRISPR-Cas9 BBi mutant and control lines using glycinin-specific antibodies. Samples in order are ‘Maverick’ (Mav., non-transgenic control); 2=Event 2; 3=Event 3; 4=Event 4; 5=Event 5; 7=Event 7; 8=Event 8.

FIG. 4A is an image showing proteomic analysis of soluble seed proteins from CRISPR-Cas9 BBi mutant and control lines using sodium dodecyl sulfate polyacrylamide gel electrophoresis of calcium fractionated soluble seed proteins visualized with Coomassie Blue; (B) immunoblot using Bowman-Birk inhibitor (BBi)-specific antibodies; (C) immunoblot using KTi-specific antibodies (KTis). Samples in order are ‘Maverick’ (Mav., non-transgenic control); 2=Event 2; 3=Event 3′ 4=Event 4′ 5=Event 5; 7=Event 7; 8=Event 8.

FIG. 4B is an image of an immunoblot analysis of CRISPR-Cas9 BBi mutant and control lines using Bowman-Birk inhibitor (BBi)-specific antibodies. Samples in order are ‘Maverick’ (Mav., non-transgenic control); 2=Event 2; 3=Event 3′ 4=Event 4′ 5=Event 5; 7=Event 7; 8=Event 8.

FIG. 4C is an image of an immunoblot analysis of CRISPR-Cas9 BBi mutant and control lines using KTi-specific antibodies (KTis). Samples in order are ‘Maverick’ (Mav., non-transgenic control); 2=Event 2; 3=Event 3′ 4=Event 4′ 5=Event 5; 7=Event 7; 8=Event 8.

FIG. 5 is a graph showing the RT-PCR analysis of T1 seeds with CRISPR-Cas9 BBi mutant and control lines.

FIG. 6 Western analysis of soybean seed proteins from RNAi-silenced BBi lines and control lines. Top panel: image of sodium dodecyl sulfate polyacrylamide gel electrophoresis (SDS-PAGE) analysis of 50% isopropanol-extracted seed proteins from T1 generation transgenic soybean plants. Seed proteins (30 μg/lane) from five individual seeds (lanes 1-5) for each of the seven independent transgenic events (ZB 168-1, ZB 168-2A, ZB 168-2B, ZB 168-4, ZB 168-5, and ZB 168-6) were separated on 15% SDS-PAGE gels and the proteins were visualized by staining with Coomassie Blue. Bottom panel: The proteins shown in the top panel were electrophoretically transferred to a nitrocellulose membrane and incubated with polyclonal antibodies specific to soybean BBi antibodies. Immunoreactive proteins were detected using anti-rabbit immunoglobulin G (IgG)-horseradish peroxidase conjugate, following chemiluminescent detection. The names of the seven transgenic events are shown at the top of the figure. PM=protein markers; May=‘Maverick’ (Mav., nontransgenic control). The numbers on the left side of the figure indicate the sizes of the protein markers in kilodaltons.

FIG. 7 Western analysis of soybean seed proteins from RNAi-silenced BBi lines and control lines. Top panel: image of an SDS-PAGE analysis of total seed proteins from T1 generation transgenic soybean plants. Seed proteins (50 μg/lane) from five individual seeds for each of the seven independent transgenic events (ZB 168-1, ZB 168-2A, ZB 168-2B, ZB 168-4, ZB 168-5, and ZB 168-6) were separated on 13.5% SDS-PAGE gels and the proteins were visualized by staining with Coomassie Blue. Bottom panel: the proteins shown in top panel were electrophoretically transferred to a nitrocellulose membrane and incubated with antibodies specific to the soybean Kunitz trypsin inhibitor. Immunoreactive proteins were detected using anti-rabbit immunoglobulin G (IgG)-horseradish peroxidase conjugate, following chemiluminescent detection. The names of the seven transgenic events are shown at the top of the figure. PM=protein markers; May=‘Maverick’ (Mav., non-transgenic control). The numbers on the left side of the figure indicate the sizes of the protein markers in kilodaltons. The arrow points to the β-subunit of β-conglycinin.

FIG. 8A is a bar graph showing results from an analysis of chymotrypsin inhibitor activity in seeds from transgenic BBi-silenced and control plants. Chymotrypsin inhibitor activity was measured at 37 °C utilizing N-Succinyl-Ala-Ala-Pro-Phe p-nitroanilide as substrate. Inhibitor activity is expressed as CIU (chymotrypsin units inhibited) per milligram of seed powder. The error bars indicate the standard error of the mean (n=3). Different letters on the top of each column indicate significant differences between the means.

FIG. 8B is a bar graph showing results from an analysis of trypsin inhibitor activity in seeds from transgenic BBi-silenced and control plants. Trypsin inhibitor activity was measured at 37 °C utilizing N-benzoyl-D,L-arginine 4-nitroanilide as substrate. Inhibitor activity is expressed as TIU (trypsin units inhibited) per milligram of seed powder. The error bars indicate the standard error of the mean (n=3). Different letters on the top of each column indicate significant differences between the means.

FIG. 9 is a bar graph showing results from an HPLC analysis of amino acid content of wild-type (Maverick) and BBi-silenced soybean (ZB168-1) seeds. Error bars represent the standard error of the mean, calculated from three biological replicates. Different lowercase letters in the same amino acid column indicate significant differences among the means (p<0.05).

FIG. 10 is an image of an SDS-PAGE analysis of in vitro protein digestibility of soybean seed proteins. Seed proteins isolated from wild-type (lanes 1, 3, 5, 7, 9, 11, and 13) and BBi-silenced soybean (ZB168-1) seeds (lanes 2, 4, 6, 8, 10, 12, and 14) were digested with trypsin and chymotrypsin. Aliquot of samples were removed at 0, 5, 10, 15, 20, 30 min, and overnight (o/n) after incubation, and separated on 13.5% SDS-PAGE gels and visualized using Coomassie Brilliant Blue staining. Note the rapid digestion of high-molecular-weight proteins and the accumulation of oligopeptides (<10 kDa) in lanes corresponding to BBi-silenced protein extracts. The numbers at the bottom of the figure refer to minutes; o/n=overnight. The numbers on the left side of the figure indicate the sizes of the protein markers in kilodaltons.

FIG. 11 is an image of an SDS-PAGE analysis of soybean seed proteins from wild type, KTI, BBi and double mutants. (A) SDS-PAGE of total seed proteins were separated on a 15% SDS-PAGE gels and the proteins were visualized by staining with Coomassie Blue; (B) Proteins shown in panel A were electrophoretically transferred to a nitrocellulose membranes and incubated separately with polyclonal antibodies specific to soybean BBi antibodies (panel B), KTi-3 antibodies (panel C), β-conglycinin antibodies (panel D) and glycinin antibodies (panel E). Immunoreactive proteins were detected using anti-rabbit immunoglobulin G (IgG)-horseradish peroxidase conjugate following chemiluminescent detection. Note the complete absence of KTi and BBi proteins in the double mutants.

FIG. 12 is an image of an SDS-PAGE analysis of soybean seed proteins from wild type, KTI, BBi and double mutants. (A) SDS-PAGE of total seed proteins were separated on a 15% SDS-PAGE gels and the proteins were visualized by staining with Coomassie Blue; (B) Proteins shown in panel A were electrophoretically transferred to a nitrocellulose membranes and incubated separately with polyclonal antibodies specific to soybean BBi antibodies (panel B), KTi-3 antibodies (panel C), β-conglycinin antibodies (panel D) and glycinin antibodies (panel E). Immunoreactive proteins were detected using anti-rabbit immunoglobulin G (IgG)-horseradish peroxidase conjugate following chemiluminescent detection. Note the complete absence of KTi and BBi proteins in the double mutants.

FIG. 13 In vitro protein digestibility of soybean seed proteins. Seed proteins isolated from wild type (Mav) and KTi/BBi double mutant (G78) seeds were digested with trypsin and chymotrypsin. Aliquot of samples were removed at 0, 5, and 30 minutes after incubation, and separated on a 13.5% SDS-PAGE gels and visualized by Coomassie Brilliant Blue staining. Note the rapid digestion of the high molecular weight proteins and the accumulation of oligopeptides (<10 kDa) in lanes corresponding to double mutant protein extracts (G78).

### DETAILED DESCRIPTION

The present inventors previously identified a soybean cell line (PI 68679) with reductions in trypsin inhibition due to a mutation affecting the KTi-1 gene was identified (Gillman et al., J. Agric. Food Chem. 2015, 63, 1352-1359). Using hybridization, the KTi-1 mutation (PI 68679) was combined with a previously identified KTi-3 mutation (PI 542044), to yield soybean lines that lack both KTi-1 and KTi-3 protein. The double KTi mutant soybean lines had lower trypsin inhibitor activity than the KTi-3 single mutant. However, all cell lines with reduced KTi levels unexpectedly also had unacceptably high levels of trypsin inhibitor activity due to overaccumulation of BBi proteins.

Naturally occurring BBi mutants have been identified in perennial soybean collections (Kollipara et al., J. Hered. 1996, 87, 461-463) and Livingston et al. reported transgenic expression of a modified BBi protein that resulted in lower BBi activity in viable plants that were able to produce seeds (Livingston et al., Plant Mol. Biol. 2007, 64, 397-408). However, no naturally occurring G. max mutant or germplasm entry has been reported with substantively reduced BBi accumulation or activity.

It is an object of the present invention to provide genetically modified soybean seeds having reduced expression of BBi proteins where the seeds have reduced trypsin and chymotrypsin inhibitor activities, as well as soybean plants, parts thereof, or cells thereof produced by growing a genetically modified soybean seed as described herein.

It is an object of the present invention to provide genetically modified soybean seeds having reduced expression of both BBi proteins and KTi proteins, where the seeds have reduced or eliminated trypsin and chymotrypsin inhibitor activities, as well as soybean plants, parts thereof, or cells thereof produced by growing a genetically modified soybean seed as described herein.

It is a further object of the present invention to provide food products for human consumption produced from the genetically modified soybean seeds, soybean plants and parts thereof having higher protein digestibility and higher amino acid adsorption, such as tofu, soy milk, plant based meat and fermented foods.

It is a further object of the present invention to provide genetically modified soybean seeds having significantly lower amounts of both trypsin and chymotrypsin inhibitor activities compared to wild-type seeds, without altering the overall protein, oil, and sulfur amino acid content of the seeds compared to wild-type.

It is a further object of the present invention to provide animal feed formulated with the genetically modified soybean seeds described herein, where heat treatment is not required during manufacture or processing of the feed.

These and additional objects of the invention are accomplished as set forth below.

Although there are at least 13 BBi genes in the soybean genome, transcriptome analysis revealed that only Glyma.16G208900 is abundantly expressed in seeds, while two others, Glyma.14G117700 and Glyma.09G158700 are expressed in seeds at relatively low levels. Extensive sequence homology shared by these seed-specific BBi genes enabled selection of conserved sequences for simultaneous knockdown of all three BBi genes. As described in more detail below, simultaneous knockout of all three seed expressed BBi genes was accomplished utilizing either a CRISPR/Cas9 or RNAi mediated approach. Both approaches resulted in stable cell lines carrying BBi loss-of-function mutations. The cells and genetically modified soybean seeds produced therefrom demonstrate substantial reductions in both trypsin and chymotrypsin inhibitor activities. In addition, overall protein, oil, and sulfur amino acid content of the seeds is not substantially different from wild-type seeds, using the “Maverick” strain as a comparator. Further, in vitro experiments show that the seed protein from the BBi knockdown lines is more rapidly hydrolyzed by trypsin and chymotrypsin, providing for enhanced protein digestibility.

### Generation and Characterization of BBi Mutants Obtained by CRISPR/Cas9 Gene Editing

CRISPR/Cas9 gene editing is now a routine practice for inducing targeted loss-of-function mutations in soybean. See e.g., Liu et al., Methods Mol. Biol. 2019, 1917, 217-234 and Sun et al., Sci. Rep. 2015, 5, 10342. Accordingly, in one aspect, the present inventors utilized a CRISPR/Cas9 system to induce mutations of soybean seed BBi genes with the aim of decreasing or eliminating the expression of the associated inhibitors in seeds and whole plants and plant products produced from the seeds, including soybeans.

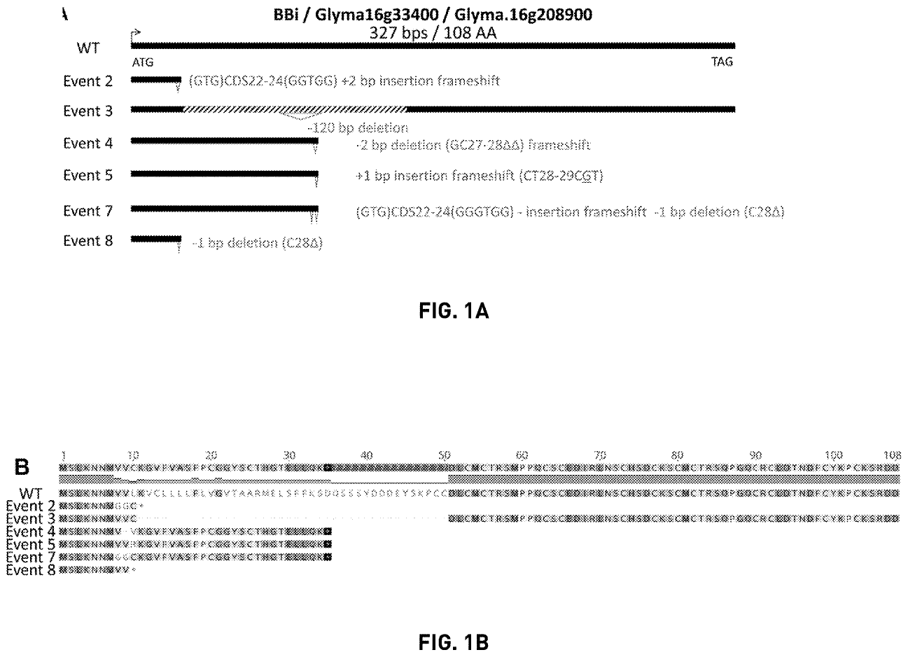

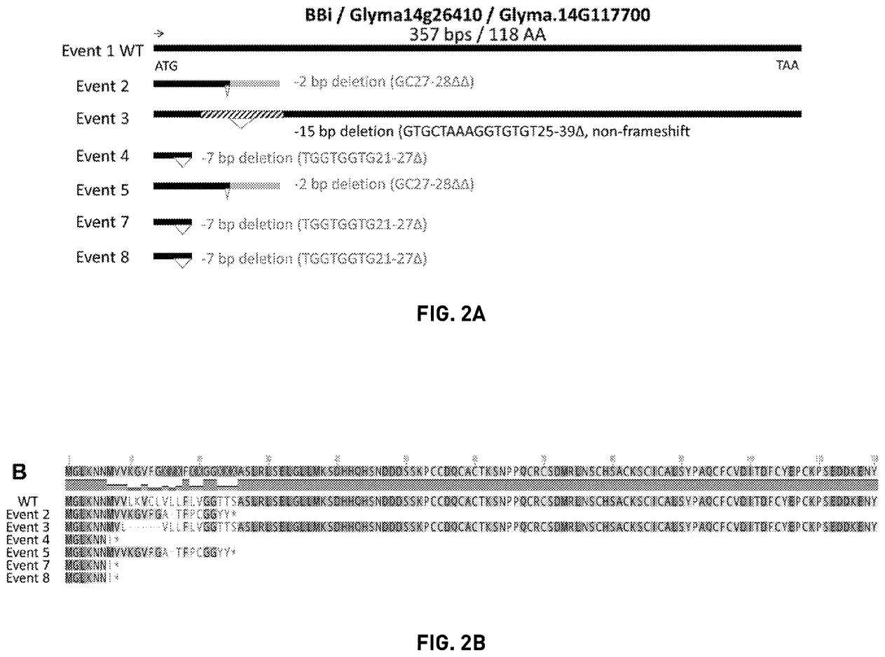

Target sites were designed to enabled simultaneous knockout of all three seed expressed BBi genes. Two CRISPR/Cas9 expression cassettes were designed, which differed in use of either a single or double guide RNA. Utilizing these two CRISPR/Cas9 expression cassettes, several independent transgenic events were generated by Agrobacterium-mediated soybean transformation (FIG. 1A). Six transgenic events were grown in the greenhouse, and leaf tissue from these plants was utilized for the isolation of genomic DNA. Sanger sequencing of PCR products generated by utilizing primers that are specific for the most abundantly expressed BBi gene (Glyma.16g208900) identified several mutations in this gene. FIG. 1A shows a schematic summary of mutations identified in these six independent events leading to deletions, insertions, and frameshift mutations on the amino acid sequence (FIG. 1). PCR products generated by utilizing primers specific for the second-most abundantly expressed BBi gene (Glyma.14G117700) were also sequenced. This analysis revealed several mutations that introduced deletions, insertions, and frameshift mutations on the amino acid sequence of the gene (FIG. 2A and FIG. 2B).

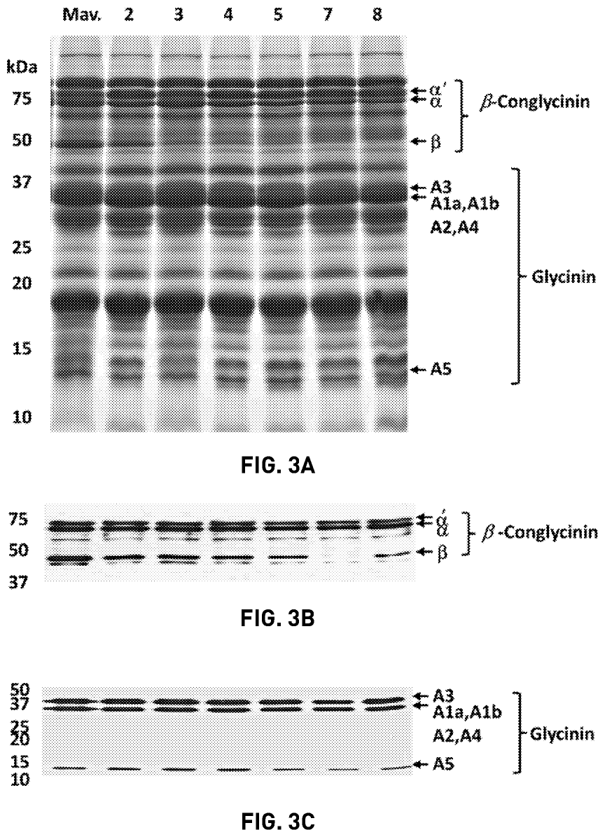

To compare the total seed protein profile between wild-type BBi mutants, total seed proteins were isolated from wild-type and the six independent transgenic events and resolved by SDS-PAGE (FIG. 3A). A visual examination of the total seed protein profile between wild-type and transgenic events did not reveal any drastic alterations between them. All soybean seeds examined accumulated the two most abundant seed storage proteins (7S β-conglycinin and 11S glycinin). However, the accumulation of the 3-subunit of β-conglycinin, a 52 kDa glycoprotein, in all transgenic events was noticeably lower when compared to the wild type. To substantiate these changes, immunoblot analysis was performed using antibodies raised against the 7S β-conglycinin and 11S glycinin antibodies. The 7S β-conglycinin antibody reacted against all three subunits. The accumulation pattern of the α′ and a subunits of β-conglycinin was similar between the wild-type and the six transgenic events (FIG. 3B). In contrast, the accumulation of the β-subunit of β-conglycinin was much lower in all transgenic events when compared to the wild type, and for one transgenic event (FIG. 3B, lane 7) only an extremely faint band was detected. Immunoblot analysis was also performed to quantify any changes in the 11S glycinin accumulation between wild-type and transgenic events. The glycinin antibody recognized three glycinin species (A3; A1a, A1b, A2, and A4; A5). In contrast to the β-subunit of β-conglycinin, the accumulation of 11S glycinin showed little variation in abundance between the wild-type and the transgenic events (FIG. 3C).

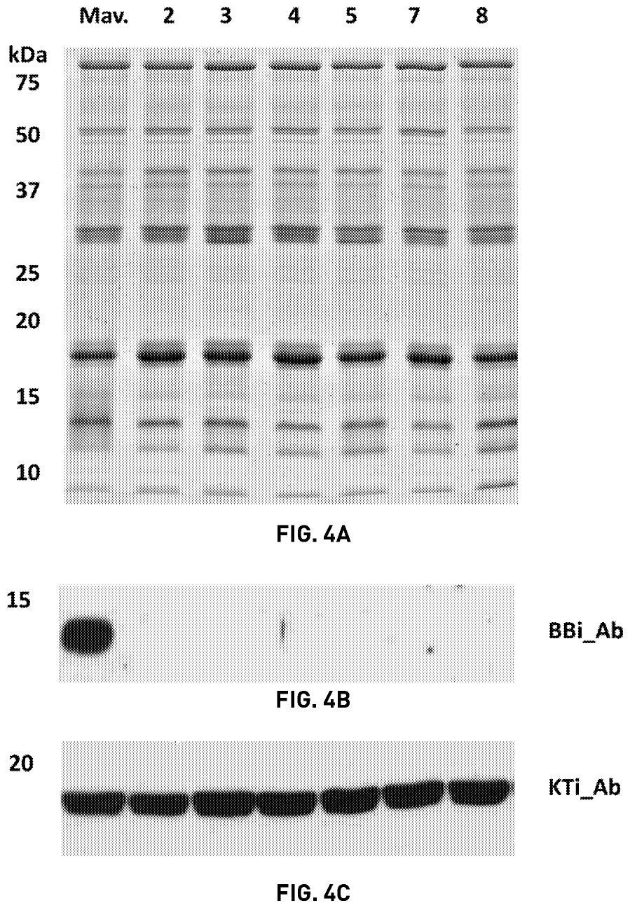

To examine the accumulation of BBi in seeds of the transgenic events, Western blot analysis was performed utilizing BBi-specific peptide antibodies (Krishnan et al., Food Chem. X 2022, 13, 100253). A protein fraction enriched for protease inhibitors obtained by the calcium fractionation method as described in Krishnan et al., Proteomics 2009, 9, 3174-3188, was utilized for the immunoblot analysis. Soybean BBi-specific antibodies failed to detect BBi protein in any of the transgenic events. In contrast, an 11-12 kDa BBi protein was detected in the wild-type plant (FIG. 4B). The accumulation of KTi, which also contributes significantly to the overall protease inhibitor of soybean seed, was also examined by immunoblot analysis, via antibodies raised against soybean KTi (Krishnan, Plant Sci. 2001, 160, 979-986). Immunoblot analysis showed normal accumulation of KTi proteins across all mutants (FIG. 4C).

Next, protease inhibitor activities were examined. Chymotrypsin inhibitor activity was measured using N-Succinyl-Ala-Ala-Pro-Phe p-nitroanilide as a substrate and expressed as CIU/mg seed powder. The wild-type soybean seed contained 17.2 CIU, while in BBi mutant lines the activity ranged from 2.89 to 15.9 CIU. Out of the six transgenic events examined in this study, four had drastic reductions; including a 2.8-6.0-fold reduction in CIU compared to the wild type (Table 1). Since soybean BBi proteins can inhibit both chymotrypsin and trypsin, trypsin inhibitor activity (TIU) was also measured using the same seed extracts, with Na-Benzoyl-DL-arginine 4-nitroanilide hydrochloride as a substrate. The trypsin inhibitor activity in the wild-type soybean seeds averaged 57.25 TIU/mg seed powder, whereas BBi mutant lines had reduced activities ranging from a low of 14.46 (Event 8) to a high of 35.7 TIU (Event 5), corresponding to a 1.6-4.0-fold reduction in TIU activity. While TIU activity was significantly lower than wild-type in all examined transgenic BBi mutant lines four (Events 2, 4, 7, and 8) lines exhibited drastic reductions ranging from 25.3 to 41.9% of the TIU activities found in wild-type seeds. Interestingly, the reduction in TIU activity in these BBi mutants is more significant than that observed for a naturally occurring soybean frameshift KTi-3 mutant. CIU and TIU activities in seeds of the mutant and the control line were fully concordant when activities were expressed as TIU/mg seed powder or as TIU/mg seed protein (Tukey HSD). The results of these analyses are summarized in Table 1.

TABLE 1
Analysis of trypsin inhibitor activity and chymotrypsin inhibitor
activities in seeds from transgenic CRISPR-Cas9BBi mutagenized
and control plants. Tukey HSD indicates the results of a Tukey-
Shapiro Honest Square Difference test; presence of the same letter
indicates insignificant differences between genotypes.

| | | Seed powder | | Seed Protein | |
|---|---|---|---|---|---|
| | | Mean ± SD | Tukey | Mean ± SD | Tukey |
| Genotype | n= | CIU/mg | HSD | CIU/mg | HSD |
| ‘Maverick’ (WT) | 3 | 17.2 ± 1.91 | A | 56.93 ± 6.32 | A |
| KTi-3 | 3 | 18.55 ± 0.42 | A | 60.97 ± 1.39 | A |
| Event 5 | 3 | 15.93 ± 1.66 | A | 56.45 ± 5.89 | A |
| Event 3 | 3 | 15.35 ± 1.63 | A | 51.34 ± 5.44 | A |
| Event 4 | 3 | 6.2 ± 1.77 | B | 21.15 ± 6.03 | B |
| Event 2 | 3 | 4.19 ± 1.03 | B | 15.28 ± 3.77 | B |
| Event 7 | 3 | 3.9 ± 1.24 | B | 16.85 ± 5.38 | B |
| Event 8 | 3 | 2.89 ± 0.04 | B | 11.54 ± 0.17 | B |

| | | Seed powder | | Seed Protein | |
|---|---|---|---|---|---|
| | | Mean ± SD | Tukey | Mean ± SD | Tukey |
| Genotype | n= | TIU/mg | HSD | TIU/mg | HSD |
| ‘Maverick’ (WT) | 3 | 57.25 ± 0.35 | A | 189.48 ± 1.17 | A |
| KTi-3 | 3 | 36.45 ± 1.63 | B | 119.8 ± 5.35 | BC |
| Event 5 | 3 | 35.7 ± 1.27 | B | 126.55 ± 4.51 | B |
| Event 3 | 3 | 31.96 ± 1 | C | 106.88 ± 3.35 | C |
| Event 4 | 3 | 24 ± 0.59 | D | 81.87 ± 2.01 | D |
| Event 2 | 3 | 21.38 ± 0.06 | DE | 77.97 ± 0.21 | D |
| Event 7 | 3 | 19.75 ± 1.89 | E | 85.35 ± 8.15 | D |
| Event 8 | 3 | 14.46 ± 1.71 | F | 57.74 ± 6.82 | E |

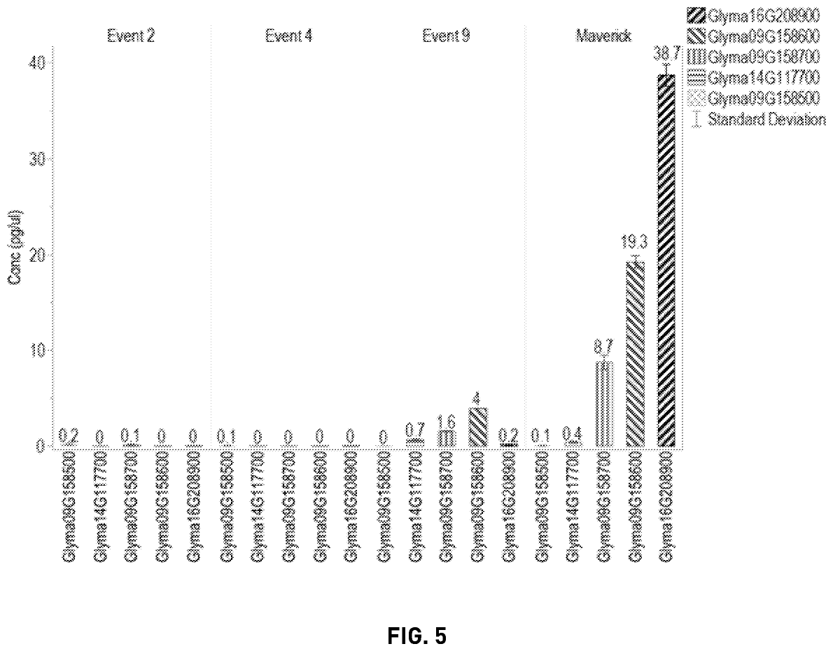

Expression of the two most abundantly expressed BBi genes (Glyma.16G208900 and Glyma. 14G117700) along with a few other BBi genes (Glyma.09G158500, Glyma.09G158600, and Glyma.09G158700) was assessed in the CRISPR/Cas9 gene-edited BBi mutants by qRT-PCR analysis. Total RNA was isolated from mid-stage (35 days after flowering) developing T1 soybean seeds and used for qRT-PCR analysis. In the wild-type soybean seeds, expression of most of these BBi genes was detected. At this stage of seed development, high level expression of Glyma.16G208900, Glyma.14G117700, and Glyma.09G158700 was observed (FIG. 5). In contrast, the expression of these abundantly expressed BBi genes was essentially not detected in two of the transgenic events (events 2 and 4), while several-fold lower expression was detected in one of the mutants (event 8).

### Generation and Characterization of BBi Mutants Obtained by RNAi Mediated Knockdown

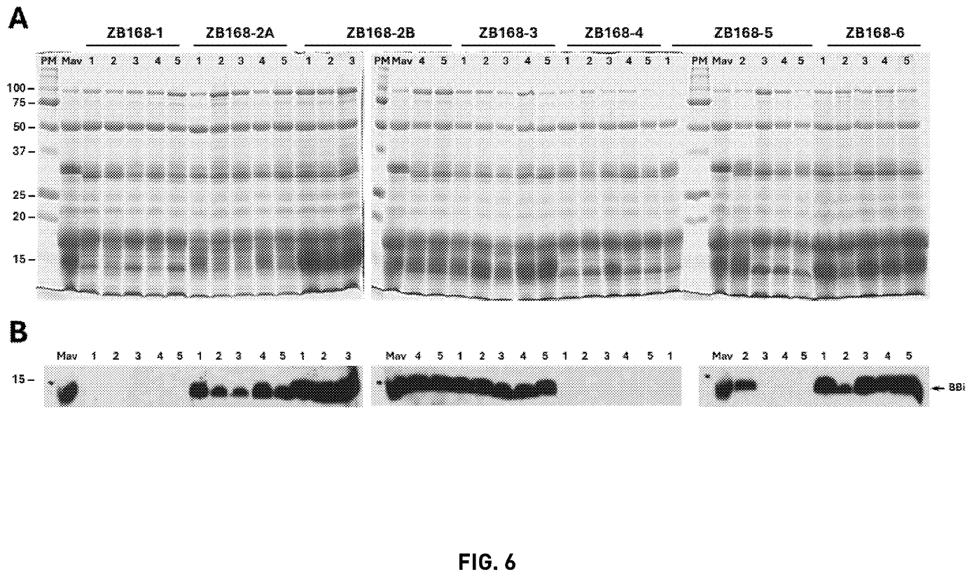

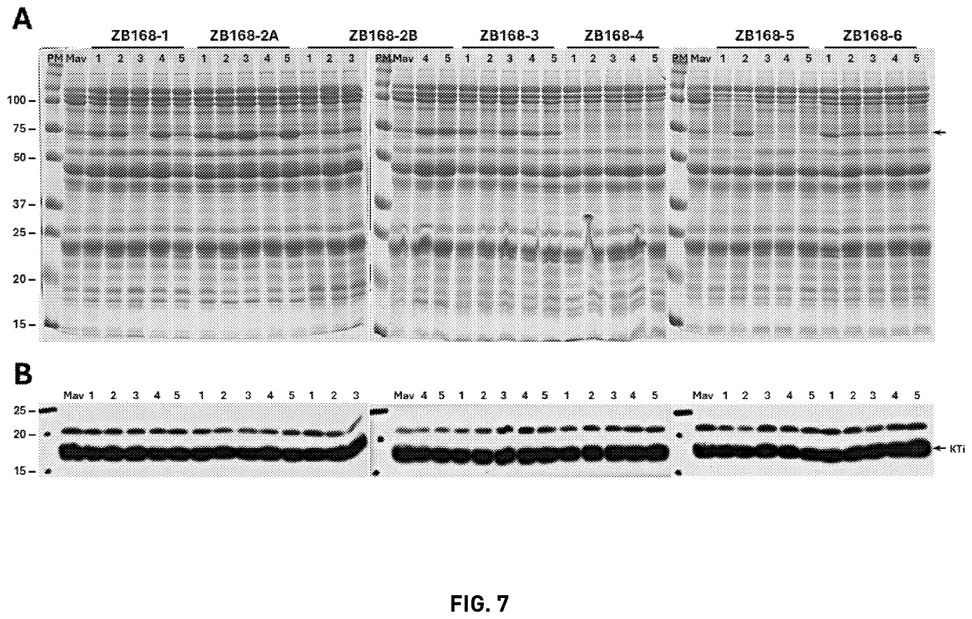

RNA interference (RNAi) technology provides an alternative methodology for introducing genetic modifications to decrease or eliminate expression of BBi genes in soybean seeds, whole plants and plant products produced therefrom. Accordingly, in another aspect, the present inventors utilized RNAi technology to knockdown the expression of BBi genes in soybean lines utilizing the Agrobacterium-mediated cotyledonary node transformation protocol, resulting in the generation of seven independent transgenic events. Seeds from these seven events were germinated and grown in a greenhouse to obtain T2 generation seeds. Seed proteins from five individual seeds, representing each of the seven transgenic events, were isolated and subjected to immunoblot analysis for the verification of the knockdown of BBi accumulation. An extract of 50% isopropanol-soluble seed proteins, previously shown to contain BBI proteins, was obtained (FIG. 6, top panel) and subjected Western blot analysis, utilizing BBi-specific polyclonal antibodies (FIG. 6, bottom panel). The analysis revealed that the BBi accumulated in three independent transgenic events, although some differences in the accumulation levels were observed. However, in one event (ZB168-5), BBi accumulation was detected in one out of the five seeds examined. In two other transgenic events (ZB168-1 and ZB168-4), no proteins cross-reacting against the BBi antibodies were detected (FIG. 6, bottom panel). An earlier study has shown that a Kunitz trypsin inhibitor mutant (KTi3) over accumulated BBi to compensate for the loss of trypsin inhibitor activity. To examine whether there is any reciprocal enhancement of the KTi due to the knockdown of the BBi, Western blot analysis was performed using the total seed proteins isolated from the dry seeds from the seven independent transgenic events (FIG. 7, top panel). Immunoblot blot analysis revealed uniform accumulation of the KTi protein in regard to all the transgenic events, even in regard to those events that failed to accumulate BBi (FIG. 7, bottom panel). An examination of the Coomassie Blue-stained total protein profile of the transgenic soybean events revealed significant differences in the accumulation of the β-subunit of β-conglycinin (FIG. 7, bottom panel, arrow).

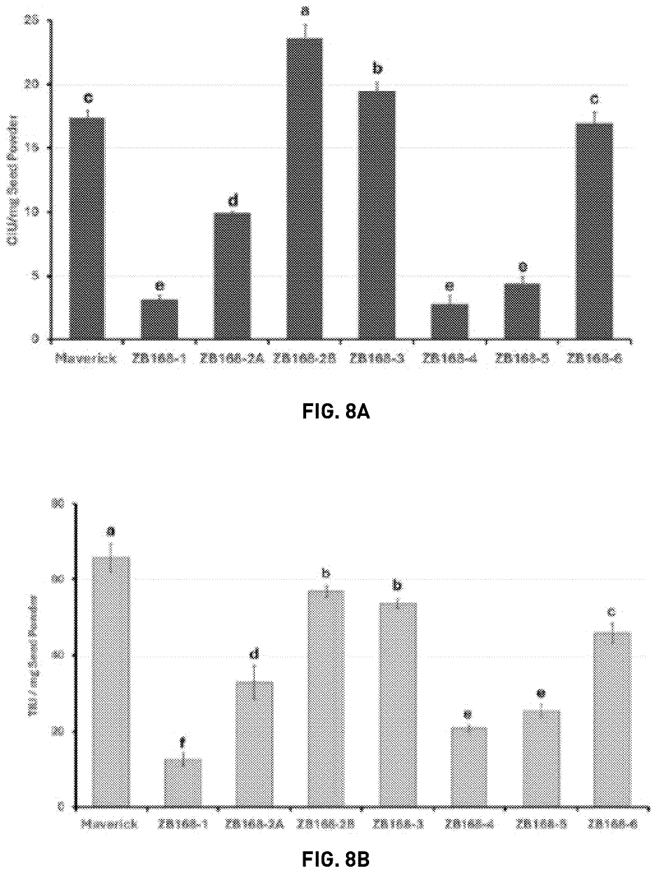

The results of the immunoblot analysis revealed the absence of BBi accumulation in some of the RNAi-silenced BBi transgenic plants. To verify whether these plants were also compromised in regard to their protease inhibitor activity, chymotrypsin inhibitor activity of the RNAi-silenced plants was measured and compared to levels found in wild-type soybean plants. The chymotrypsin inhibitor activity (CIU) was measured using N-Succinyl-Ala-Ala-Pro-Phe p-nitroanilide as a substrate, expressed as the CIU per mg of seed powder. The wild-type soybean seeds contained 17.2 CIU, while the BBi-mutated transgenic seeds showed activity ranging from 2.89 to 15.9 CIU. Out of the seven transgenic events examined, four exhibited drastic reductions, with a 2.8-6.0-fold decrease in the CIU compared to the wild type (FIG. 8A). The chymotrypsin inhibitor activity in the seeds for four transgenic events (ZB168-1, ZB168-2A, ZB168-4, and ZB168-5) were significantly lower than the wild-type seeds. In regard to the transgenic events, ZB168-1 and ZB168-4, the chymotrypsin inhibitor activity was reduced by greater than 80% compared to the wild-type seeds. In regard to the other two transgenic events (ZB168-5 and ZB168-2A), the chymotrypsin inhibitor activity was reduced by 75% and 45%, respectively (FIG. 8A). In contrast, the chymotrypsin inhibitor activity was not impacted in regard to transgenic event ZB168-6, while slightly elevated activity was detected in regard to transgenic event ZB168-2B and ZB168-3 compared to wild-type seeds (FIG. 8A).

Since the soybean BBi can inhibit both chymotrypsin and trypsin, trypsin inhibitor measurements were also performed, using Na-Benzoyl-L-arginine 4-nitroanilide hydrochloride as a substrate. The trypsin inhibitor activity was expressed as the TIU per mg of seed powder. The wild-type soybean seeds averaged 57.25 TIU per mg of seed powder, while, in the BBi-silenced transgenic plants, the trypsin inhibitor activity ranged from 14.46 to 35.7 TIU (FIG. 8B). Out of the seven transgenic events, four events, ZB168-1, ZB168-2A, ZB168-4, and ZB168-5, revealed significantly lower trypsin inhibitor activity compared to wild-type plants. The trypsin inhibitor activity in other transgenic lines (events ZB168-2B, ZB168-3, and ZB168-6) was only marginally reduced compared to wild-type seeds. The seeds from three transgenic events (ZB168-1, ZB168-4, and ZB168-5) that failed to accumulate BBi proteins and showed significantly lower trypsin and chymotrypsin inhibitor activities were advanced to T3 and T4 generation. The absence of the BBi in the T3 and T4 generation was confirmed by immunoblot analysis, using BBi-specific polyclonal antibodies. For all further analysis, seeds from the T4 generation of ZB168-1 were employed.

Immunocytochemical localization of the BBi was performed by incubating thin sections of paraffin-embedded soybean seeds first with BBi-specific antibodies, followed by Alexa Fluor 488-labeled goat anti-rabbit antibodies. When thin sections of wild-type soybean seeds were examined under a scanning confocal microscope, bright green fluorescence was observed in all the parenchymatous cotyledonary cells. Interestingly, fluorescence was primarily restricted to the cytoplasm. No specific localization of the BBi was detected in the protein storage vacuoles, which are the main storage organelles for the abundant seed storage proteins of soybeans. An examination of the thin sections of BBi knockdown soybean seeds revealed a drastic reduction in the intensity of green fluorescence in the parenchymatous cotyledonary cells, indicating very low accumulation of BBi in the transgenic seeds.

To determine whether the silencing of BBi genes resulted in any changes in the transcriptome of soybean seeds RNA-seq analysis was performed at three development stages. The raw sequencing reads were trimmed to remove possible adapter sequences and nucleotides with poor quality, before being mapped to the Glycine_max_Wm82a4v1 reference genome map. This analysis revealed a mapping efficiency ranging between 62 and 63% for young, 82-91% for mid-stage, and 91-94% for late seed developmental stages. Using expression values from each sample, a sample-sample distance heatmap was constructed showing the clustering of gene expression profiles between wild-type and BBi-silenced soybean seeds (ZB168-1) for all three developmental stages. Principal component analysis (PCA) of the RNA-seq data revealed a high similarity between the three biological replicates at each of the three seed developmental stages.

A comparison of gene expression between the RNAi-derived BBi mutant (ZB168-1) and the wild type (‘Maverick’ soybean) was performed. For the visual identification of genes with large fold changes that are statistically significant, Volcano plots were constructed. Genes with an adjusted p-value of less than 0.05 and a log 2 fold change greater than 1 were included. Similarly, genes with an adjusted p-value of less than 0.05 and a log 2 fold change greater than −1 were also included. In these plots, the most upregulated genes in the RNAi-derived BBi mutant are shown on the right, while the most downregulated genes are on the left side of the plot. During the early stages of seed development, 318 genes were found to be downregulated and 167 genes were upregulated. At early-stage seed development, a limited number of genes in the RNAi-derived BBi mutant were either upregulated or downregulated. The upregulated genes included five genes (Glyma.15G119800, Glyma.15G119700, Glyma.15G199600, Glyma.20G148200, and Glyma.18G157800) with a log fold change greater than 1. Similarly, three genes (Glyma.16G208900, Glyma.08G087100, and Glyma.19G178100) were downregulated. At the mid-stage of seed development, there were 2539 DEGs, out of which 1119 genes were upregulated and 1420 genes were downregulated. During the later stages of seed development, a greater number of DEGs between the wild-type and CRISPR-BBi knockdown lines were observed. At this stage, there were 3347 DEGs, out of which 1284 genes were upregulated, and 2063 genes were downregulated. Since the number of DEGs was relatively large, a stringent filtering threshold was applied to obtain a list of highly correlated DEGs. By applying a stringent filtering threshold of |log 2FC|≥3 and p-value<0.01, genes located at the extreme ends of the volcano plot were obtained.

As noted above, the soybean genome contains at least 13 different BBi genes, out of which only a few of them are expressed in appreciable levels in soybean seeds. Transcriptomic analysis has shown that Glyma.16g208900 encodes the most abundantly expressed BBi gene in developing soybean seeds, followed by Glyma.14G117700. The other BBi genes that are expressed, albeit at very low levels, are Glyma.09G158700, Glyma.09G158600, Glyma.09G158800, and Glyma.09G158500, respectively. The expression pattern of these six seed expressed BBi genes was examined in wild-type and BBi-silenced seeds. All the BBi genes were expressed in wild-type soybean seeds, with the highest levels of expression detected at the mid-stage of development. As was reported earlier, Glyma.16g208900 was the most abundantly expressed BBi gene in wild-type soybean seeds. However, the expression of this gene was not detected in BBi-silenced transgenic seeds. Interestingly, low levels of expression of other BBi genes (Glyma.14G117700, Glyma.09G158700, Glyma.09G158600, Glyma.09G158800, and Glyma.09G158500) was detected in BBi-silenced transgenic seeds, although their expression levels were significantly reduced compared to the wild-type seeds. Previous work has shown that BBi-silenced soybean seeds contained reduced levels of trypsin inhibitor activity compared to that of the wild-type seeds. Accordingly, differences in the expression levels of KTi genes in wildtype and BBi-silenced soybean seeds was evaluated. There are three KTi genes (KTi1, KTi2, and KTi3) in soybeans, with KTi3 being the most abundantly expressed gene in soybean seeds. A comparison of the expression levels of the KTi genes revealed that the expression of all three KTi genes were almost similar at the early seed developmental stage between the wild-type and BBi-silenced soybean seeds. The expression of the KTi2 gene peaked during the early developmental stage, declined at the mid-stage, and was undetectable by the late stage. In contrast, the expressions of KTi1 and KTi3 were abundant at the mid-stage and declined at the later stages. Although a significant decrease in KTi1 and KTi3 expression was observed in BBi-silenced soybean seeds compared to the wild type at the mid-stage of development, their expression levels were similar at the later stages of seed development.

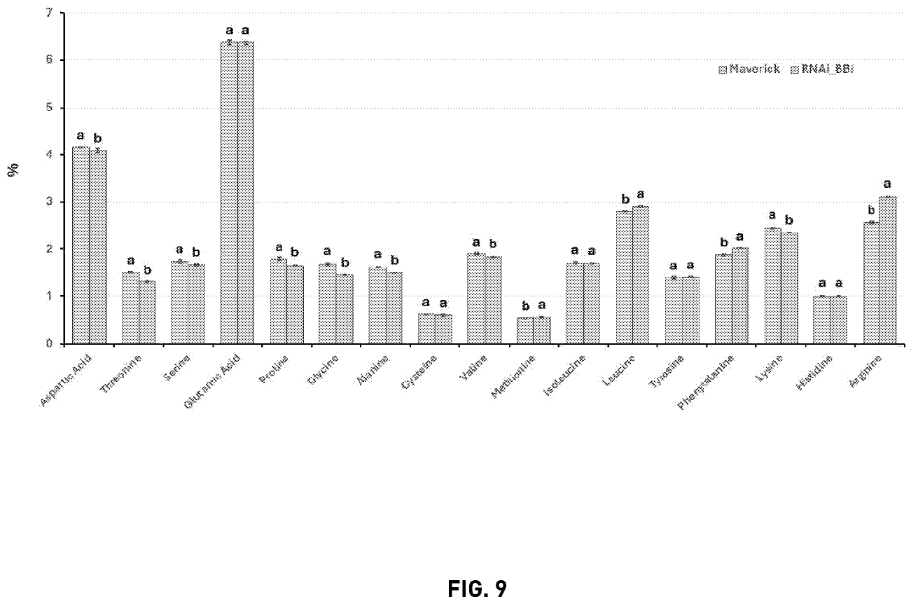

BBi is a sulfur-rich protein, characterized by the presence of multiple cysteine residues. To investigate whether silencing BBi genes affects the sulfur-containing amino acid content, we conducted amino acid profiling, using high-performance liquid chromatography (HPLC) (FIG. 9). Overall, the amino acid compositions were largely consistent across the wild-type and knockdown seeds. Nevertheless, a one-way ANOVA, followed by Tukey's HSD post hoc test (p<0.05), indicated significant differences in the levels of several individual amino acids. Notably, the cysteine levels remained statistically unchanged between the two genotypes. Interestingly, the methionine content showed a slight increase in the BBi knockdown line (0.55%) compared to the wild type (0.53%). Protein and oil content of the seeds was also measured, using near-infrared reflectance (NIR) spectroscopy. The protein and oil contents of the wild-type soybean or Maverick were 40.49±0.42% and 20.01±0.48%, respectively. Similarly, the protein and oil contents of BBi-silenced soybean seeds were also 40.49±0.18% and 20.01±0.12%, respectively. These results indicate that despite the pronounced downregulation of BBi expression, there were no appreciable differences in the seed protein, oil content, or sulfur amino acid composition. These findings suggest that BBi gene disruption exerts minimal influence on seed nutritional quality and composition in soybean.

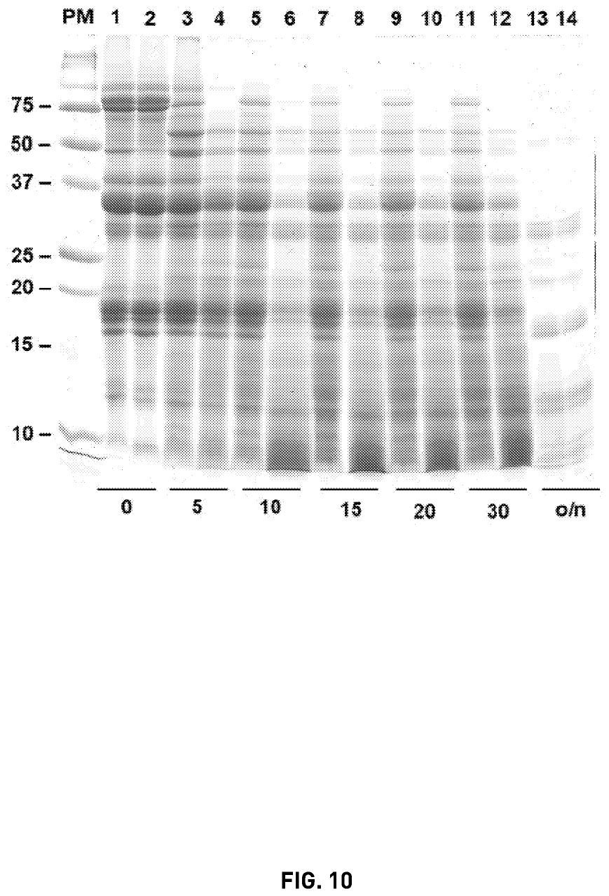

Since BBi inhibits both of the digestive enzymes, trypsin and chymotrypsin, experiments were undertaken to examine whether the reduction in the BBi activity had any effect on protein digestibility. An examination of the SDS-PAGE-resolved protein profiles at different times of digestion with trypsin and chymotrypsin revealed that the seed proteins in the BBi knockdown lines were much more rapidly digested compared to that of wild-type seed proteins (FIG. 10). The increased digestibility of seed proteins can be observed within 5 min after incubation with trypsin and chymotrypsin. The appearance of low molecular peptides at the bottom of the SDS-PAGE gels indicates that the lack of BBi has resulted in the increased susceptibility of soybean seed proteins to trypsin and chymotrypsin.

### Generation and Characterization of KTi and BBi Double Mutants

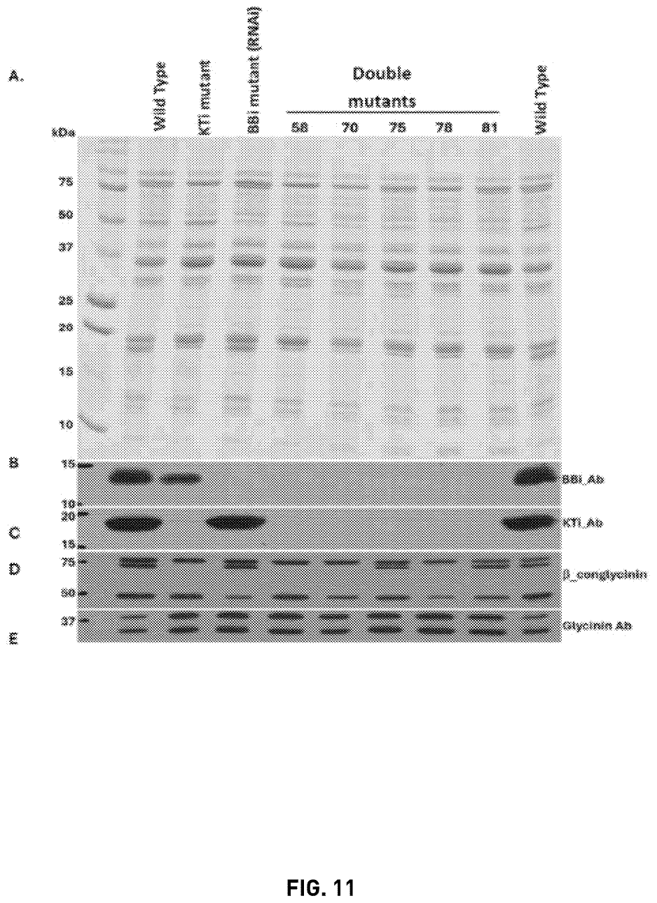

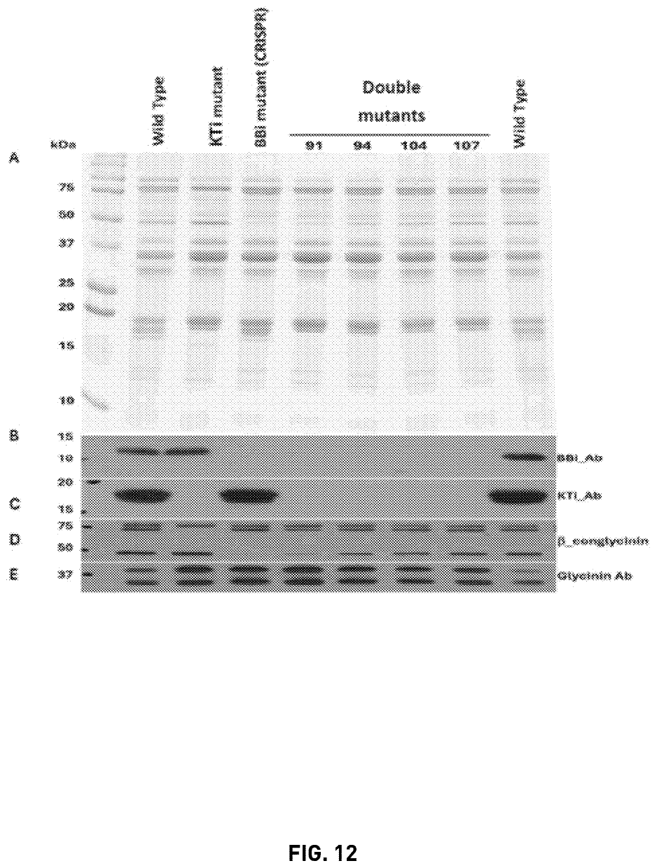

Double mutants containing knockdowns of both KTi-3 and BBi were generated via hybridization of two KTi mutant strains with the BBi mutants described above. Briefly, a naturally occurring KTi-3 mutant (PI 157740) exhibits a substantial reduction (~40%) in trypsin inhibitor activity. Through germplasm screening, a second KTi-1 mutant (PI 68679) was identified. These two mutant lines were crossed through conventional hybridization, and the resulting progeny were advanced to generate an experimental soybean line (E-16) deficient in both KTi-1 and KTi-3 proteins. To generate soybean lines simultaneously lacking KTi and BBi proteins, the E-16 line was crossed with either an RNAi-mediated BBi knockdown line (FIG. 11) or a CRISPR/Cas9-derived BBi mutant (FIG. 12). Seeds from these crosses were germinated and grown in the greenhouse to obtain the F₂ generation, which was subsequently advanced to the F₃ generation. Seed proteins from individual F₃ seeds were extracted and analyzed by immunoblotting to confirm the absence of KTi and BBi proteins. Immunoblot results demonstrated that both inhibitors were completely eliminated in the KTi/BBi double mutant lines (FIG. 11 and FIG. 12).

To determine whether the KTi/BBi double mutants were fully compromised in protease inhibitor activity, both trypsin and chymotrypsin inhibitor activities were quantified and compared to wild-type soybean seeds. Chymotrypsin inhibitor activity (CIU) was measured using N-Succinyl-Ala-Ala-Pro-Phe p-nitroanilide as the substrate and expressed as CIU per mg of seed powder. The results are summarized in Table 2 below. Chymotrypsin and trypsin inhibitor activities were measured at 37 °C utilizing N-Succinyl-Ala-Ala-Pro-Phe p-nitroanilide and N-benzoyl-D,L-arginine 4-nitroanilide as substrates, respectively. Inhibitor activities are expressed as CIU (chymotrypsin units inhibited) and TIU (trypsin units inhibited) per milligram seed powder.

TABLE 2
Analysis of chymotrypsin inhibitor activity and trypsin inhibitor
activity in seeds of wild type, KTI, BBi and double mutant plants.

| cultivar ID | TIU/mg seed powder | CIU/mg seed powder |
|---|---|---|
| WT | 54.21 +/− 1.83 | 16.18 +/− 2.01 |
| KTi-mutant | 38.81 +/− 6.98 | 17.63 +/− 3.87 |
| BBi-mutant | 13.96 +/− 4.64 | 5.02 +/− 0.45 |
| G78 Double mutant | 0 | 0 |

Wild-type seeds exhibited 16.18 CIU, whereas the KTi and BBi single mutants showed 17.63 and 5.02 CIU, respectively. Notably, the double mutant displayed no detectable chymotrypsin inhibitor activity. Trypsin inhibitor activity was assessed using Na-Benzoyl-L-arginine 4-nitroanilide hydrochloride as the substrate and expressed as TIU per mg of seed powder. Wild-type seeds averaged 54.21 TIU per mg of seed powder, while the KTi and BBi single mutants showed 38.81 and 13.96 TIU, respectively. As with chymotrypsin inhibition, the double mutant exhibited no measurable trypsin inhibitor activity.

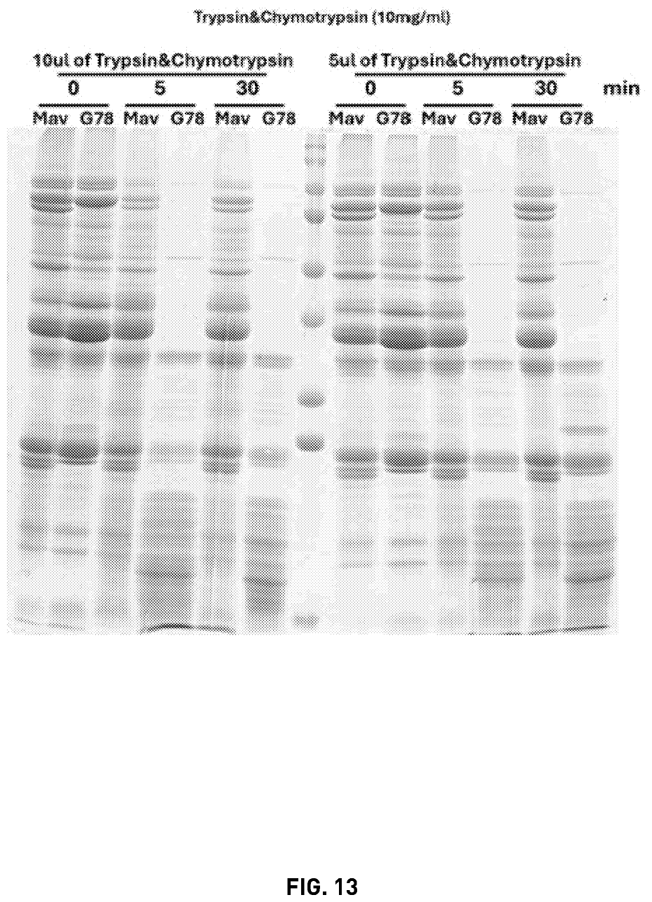

SDS-PAGE analysis of seed protein extracts subjected to in vitro digestion with trypsin and chymotrypsin revealed that proteins from the KTi/BBi double mutant line (G78) were degraded much more rapidly than those from wild-type seeds (FIG. 13). Enhanced digestion was evident as early as 5 minutes after incubation with the proteases. The rapid disappearance of major seed storage proteins, accompanied by the accumulation of low-molecular-weight peptides near the bottom of the gel, indicates that the absence of KTi and BBi substantially increases the susceptibility of soybean seed proteins to proteolytic cleavage. These results demonstrate that eliminating both inhibitors significantly improves protein digestibility in soybean seeds.

### DISCUSSION

The data provided here demonstrates the successful generation of several stable genetically modified soybean lines exhibiting advantageously low levels of trypsin and chymotrypsin inhibitor activities. These soybean lines may be useful for improving the formulation of animal feed due to their significantly reduced levels of trypsin and chymotrypsin inhibitors, two key antinutritional factors that typically require extensive heat treatment in order to be neutralized. By reducing the levels of these protease inhibitors, the genetically modified lines offer several advantages. First, they are likely to require minimal heat processing, translating into substantial cost savings during production. Additionally, the absence of protease inhibitors enhances protein digestibility, thereby improving nutrient utilization and boosting animal performance. The results of the in vitro protein digestion analysis further underscore the benefits of the genetically modified lines. Proteins derived from these soybeans are broken down more efficiently into lower molecular weight oligopeptides compared to those from wild-type soybeans. This improved digestibility highlights the potential of the genetically modified soybeans described herein as a superior ingredient in animal feed, offering both economic and nutritional benefits, as well as food for human consumption having higher protein digestibility and higher amino acid adsorption.

Until now, no soybean null mutants with reduced or ablated BBi proteins have been identified in G. max germplasm. While BBi mutants have been identified in several perennial Glycine spp., substantial hybridization barriers exist between perennial Glycine spp. and G. max, which has hampered the development of G. max BBi mutants by traditional breeding. Attempts have been made to lower the protease inhibitor activity in soybean seeds through overexpression of a mutated BBi gene (Livingstone et al., Plant Mol. Biol. 2007, 64, 397-408). The transgenic seeds generated in that study revealed a 20-50% reduction in protease inhibitory activity. In contrast, several of the BBi mutant lines described here showed remarkable reduction (ranging from 63 to 84%) in chymotrypsin inhibitor activity when compared to the wild-type seeds. Even though the CRISPR-Cas9 constructs were targeted to knockout seed-specific BBi genes, their pronounced effect on lowering the trypsin inhibitor is striking. This drastic reduction in trypsin inhibitor activity is not mediated by a reduction in the accumulation of KTi proteins in the BBi mutant lines, since immunoblot analysis clearly demonstrates that KTi proteins accumulate to similar levels in BBi mutant lines as in wild-type seeds. Thus, despite the presence of functional versions of all three KTi seed-expressed genes, there was a significant reduction in the trypsin inhibitor activity, unexpectedly revealing that BBi contributes significantly to overall trypsin inhibitor activity in soybean seeds. The immunoblot analysis and qRT-PCR analysis confirmed that the gRNA was very effective in knocking out the seed-specific BBi genes. In addition to eliminating the expression of the two most abundant BBi genes (Glyma.16G208900 and Glyma.14G117700), other BBi genes (Glyma.09G158500, Glyma.09G158600, and Glyma.09G158700) were also eliminated in some of the mutant lines.

The results discussed above also provide RNAi-generated BBi knockdown soybean lines having significantly reduced chymotrypsin and trypsin inhibitor activity. Western blot analysis confirmed that the BBi knockdown lines failed to accumulate this protein in their seeds, while transcriptomic analysis revealed a substantial downregulation of most seed-specific BBi genes in the knockdown lines. However, the suppression of the most abundant seed-specific BBi genes did not eliminate chymotrypsin inhibitor activity. All the transgenic soybean lines generated in this study exhibited residual chymotrypsin inhibitor activity, albeit at much lower levels than wild-type soybeans. The residual chymotrypsin inhibitor activity may be due to the upregulation of minor seed-specific BBi genes. Supporting this hypothesis, transcriptomic analysis revealed that two BBi genes, Glyma.18G231400 and Glyma.18G231500, which are expressed at extremely low levels in wild-type seeds, exhibited increased expression in the BBi knockdown line. This upregulation could account for the residual chymotrypsin inhibitor activity observed in the BBi knockdown lines.

Finally, the results presented here demonstrate the successful generation of double mutants containing knockdowns of both KTi-3 and BBi, confirming it is possible to eliminate both trypsin and chymotrypsin inhibitor activities in soybean seeds.

### Materials and Methods

Chemicals: Most chemicals and reagents used in this study were of analytical grade. Chemicals for electrophoresis, including acrylamide, bis acrylamide, SDS, TEMED, and ammonium persulfate, were purchased from GE healthcare (Piscataway, NJ, USA). Tris-HCL, trypsin, chymotrypsin, β-mercaptoethanol, Na-Benzoyl-DL-arginine p-nitroanilide hydrochloride, and N-Succinyl-Ala-Ala-Pro-Phe p-nitroanilide were purchased from Sigma-Aldrich (St. Louis, MO, USA).

Analysis of Expression of BBi Genes Using Public Data Repositories: Soybase.org is a repository for data from multiple experimental datasets for soybean. Protein coding genes were identified in the ‘Williams 82’ genome soybean with the annotation PFAM PF00228 (Bowman-Birk serine protease inhibitor family) as targets. Previously collected data from the ‘RNA-Seq Atlas’ (Severin et al., BMC Plant Biol. 2010, 10, 160) was used to prioritize target BBi genes for CRISPR-Cas9 constructs, gene sequencing, and RT-PCR analysis. In developing seeds, two genes, Glyma.14g117700 and Glyma.16g208900, represent between 17.0 and 31.3% and 68.5 and 82.8% of the total detectable BBi gene expression from seeds samples between 21 and 42 days after flowering.

Generation of CRISPR/CAS9-Induced BBi Knockout Soybean Mutants: For CRISPR/Cas9-mediated mutagenesis of the BBi genes in soybean, two single gRNAs were designed to target the two major expressed BBi genes and three other BBi genes. gRNA1 was a common target in five BBi genes (Glyma.09G158500; Glyma.14G117700; Glyma.09G158600; Glyma.09G158700; Glyma.16G202800), and gRNA2, reverse complimentary of the sense strand sequence was a common target in only 3 BBi genes (Glyma.09G158600; Glyma.09G158700; Glyma.16G202800). Two plasmid vectors were constructed. The first plasmid contained only gRNA1, and the second plasmid contained both gRNA1 and gRNA2. For cloning of the BBi gRNAs in the T-DNA vector, a two-step Golden Gate Assembly was performed firstly in the module vector pMOD_B2103 and then subsequently in the destination T-DNA vector pTRANS230d as described previously (Liu et al., Methods Mol. Biol. 2019, 1917, 217-234 and Cermak et al., Plant Cell 2017, 29, 1196-1217). Recombinant pMOD_B2103_BBi gRNAs plasmids harboring BBi gRNAs were verified by Sanger sequencing of the gRNA cassette with primers flanking the gRNAs: the forward primer TC320 and reverse primer TC089R, respectively. The second Golden Gate Assembly reaction was composed of the recombinant pMOD_B2103_BBi gRNAs, pMOD_A0521, pMOD_C2906, and a destination T-DNA vector pTRANS230d. Final CRISPR/Cas9_BBi gRNA constructs in the destination T-DNA vector were designated as pTRANS230/BBi_1gRNA and pTRANS230/BBi_2gRNA, respectively. The CRISPR constructs were mobilized into a disarmed Agrobacterium tumefaciens strain EHA105 by a heat shock procedure.

Generation of BBi Mutants by RNAi-mediated knockdown: To simultaneously suppress the expression of Bowman-Birk inhibitors (BBi), an RNAi cassette was constructed following the procedure described in Kim et al., Int. J Mol. Sci. 2025, 26, 6943. The RNAi construct was mobilized into a disarmed Agrobacterium tumefaciens strain, EHA105, via heat-shock transformation. Soybean transformation was performed following the protocol described below. ‘Maverick’ soybean (Reg. no. CV-372, PI 598124), which was developed by the Missouri and Illinois Agricultural Experiment Stations at the Universities of Missouri and Illinois, respectively, was used for plant transformation. Putative transgenic soybean plants were identified by a leaf paginating assay, using 100 mg of L-1 glufosinate ammonium solution on leaves at three stages. Herbicide-resistant plants were then cultivated in greenhouses under controlled conditions, maintaining a 16 h day at 28 °C and an 8 h night at 24 °C.

Soybean Transformation: The soybean genotype ‘Maverick’ (Sleper et al., Crop Sci. 1998, 38, 549-550) was transformed using Agrobacterium-mediated transformation of half-seed explants. The transformation protocol was modified from Zeng et al. Plant Cell Rep. 2004, 22, 478-482. The process began by germinating sterilized seeds for 16 h in Petri dishes on germination media. Then, half seed explants were made from the seeds and treated with an Agrobacterium culture (Paz et al., Plant Cell Rep. 2006, 25, 206-213). After 5 days of co-cultivation, the explants were transferred to shoot induction medium. Subsequently, the explants underwent two rounds of subcultures before being moved into shoot elongation medium. Elongated shoots were then cut from the explants and used for rooting. After 2 weeks, the rooted plants were transferred into soil. To identify putative transgenic soybean plants, a leaf paginating assay was performed with 100 mg L-1 glufosinate-ammonium solution on leaves at three stages. Herbicide-resistant plants were cultivated in greenhouses under controlled conditions, with a 16 h day at 28 °C and 8 h night at 24 °C until T1 seeds were harvested.

Protein Extraction and 1-D Electrophoresis: Total soybean seed protein extraction and subsequent separation of proteins by 1-D SDS-PAGE were carried out as previously described (Song et al., J. Agric. Food Chem. 2018, 66, 432-439). Briefly, dry soybean seeds were ground to a fine power with a mortar and pestle and extracted with 1 mL of sodium dodecyl sulfate (SDS) sample buffer (60 mM Tris-HCl, pH 6.8, 2% SDS, 10% glycerol and 5% 2-mercaptoethanol). Tubes were vortexed (Vortex Genie, Scientific Industries, San Diego, CA, USA) for 10 min at room temperature and then heated to 100 °C for 5 min followed by centrifugation (Eppendorf, Beckman Coulter, Inc., Indianapolis, IN, USA) at 15,800×g for 5 min. The supernatant is the total seed protein fraction. This fraction was resolved by 13.5% SDS-PAGE gels using a Hoefer SE 260 minigel apparatus (Amersham Biosciences, Piscataway, NJ, USA). Electrophoretic separation of proteins was achieved with 20 mA per gel (constant current). SDS-PAGE-separated proteins were visualized using Coomassie Blue R-250 solution (0.3% Coomassie Brilliant Blue R-250, 45% methanol, and 10% glacial acetic acid).

Immunoblot Analysis: The proteins were extracted from the wild-type soybean cultivar ‘Maverick’ and T1:2 seeds of the BBi-mutated lines were separated using 1³0.5% SDS-PAGE gels. These proteins were then transferred to nitrocellulose membranes and incubated with TBS (10 mM Tris-HCl, pH 7.5, 500 mM NaCl), supplemented with 5% non-fat dry milk for 1 h at room temperature. After incubation, the nitrocellulose membranes were washed three times with TBST (15 min each) and incubated overnight with antibodies at a 1:10,000 dilution, specifically raised against either purified soybean Kunitz trypsin or Bowman-Birk protease inhibitors (Krishnan et al., Crop Sci. 2012, 52, 1786-1794). The 7S β-conglycinin and 11S glycinin-specific antibodies were used at 1:50,000 dilution. Specifically bound primary antibodies were detected using anti-rabbit IgG-horseradish peroxidase conjugates and the SuperSignalWest Pico kit (Pierce, Rockford, IL, USA).

Real-Time qRT-PCR Analysis of BBi Genes in Developing Soybean Seeds: Total RNA from 35 days after anthesis soybean seeds was extracted using TRIzol™ reagent (Invitrogen, Waltham, MA, USA) according to the manufacturer's instructions. Real-time qRT-PCR was conducted using a LightCycler™ 480 II instrument (Roche, Indianapolis, IN, USA). Each qRT-PCR reaction was conducted with two replications in 20 μL using the QuantiNova SYBR Green RT-PCR Kit (Qiagen, Hilden, Germany) following the manufacturer's instructions. The cycling condition of absolute quantification analysis were 10 min at 50 °C then 2 min 95 °C for cDNA synthesize followed by 40 cycles of 20 s at 95 °C and 40 s at 60 °C, with the data acquired at 60 °C in the green channel. The reference gene (F-box protein, Glyma.12G051100) was amplified by RT-PCR from Maverick soybean seed total RNA with a gene-specific primer set. The amplified reference gene RT-PCR fragment was purified from agarose gel, and the standard was calculated based on the purified fragment concentration and molecular weight. The reference gene was subjected to a 10-fold serial dilution (1.00×101 to 1.00×10-10) with DEPC. For absolute quantification analysis, five standard dilutions from 1.00×10-5 to 1.00×10-9 for a standard curve were used for quantifying both target and reference gene expression in cDNA samples.

Screening of Putative Transgenic Plants and Identification of CRISPR-CAS9-Induced Mutations: Twenty seeds from 10 individual TO BBi events were planted in the Sears Plant greenhouse in 2-gallon pots containing Pro-Mix® HP Mycorrhizae™ (Premier Tech, Quakertown, PA, USA). The plants were fertilized with Osmocote Plus (Scotts, Marysville, OH, USA) according to the manufacturer's recommendations. Greenhouse settings were 16 h daylength and 30 °C/18 °C day/night temperatures. For every plant that germinated, fresh tissue was collected, and genomic DNA isolated from ~20-30 mg lyophilized leaf tissue using a Maxwell Promega robot with the Maxwell RSC PureFood GMO and Authentication Kit (Promega, Madison, WI, USA). PCR primer pairs were designed to bear at least two gene-specific SNP differences as compared to homologous genes. The sequences of all primers were compared to the unmasked Glycine max ‘Williams 82’ genomic sequence (E-value cut off=10.0) to ensure primer specificity (www.soybase.org, accessed on 15 Nov. 2021). PCR amplification was performed using Titanium taq according to the manufacturer's recommendations (Takara, Kusatsu, Japan) in a PTC-200 thermocycler (MJ Research/Bio-Rad, Hercules, CA, USA), using these conditions: 95 °C for an initial 5 min denaturation, followed by 40 cycles of 95 °C for 30 s, followed by 60 °C for 30 s, and an extension step at 72 °C for 1 min/kilobase of the target sequence. PCR products were separated on a 1% agarose gel and compared to DNA ladder size standards to ensure appropriate size. PCR products were purified through use of a QIAquick PCR purification kit (Qiagen, Hilden, Germany). After PCR purification, products were Sanger-sequenced at the DNA Core Facility of the University of Missouri-Columbia. PCR product sequencing traces were imported into Geneious Primer software, version 11.0.11+9 (Invitrogen); trimmed; and aligned to gene sequences from the G275_W82.a2.v1 assembly (http://www.phytozome.net/soybean, accessed on 12 Feb. 2022) using the Geneious Primer software, version 11.0.11+9 (Invitrogen). Disagreements were manually assessed in comparison to the G275_W82.a2.v1 assembly (Schmutz et al., Nature 2010, 463, 178-183). Putative indels and polymorphisms were verified by a minimum of two independent PCR reactions.

Trypsin and Chymotrypsin Inhibitor Assay: Trypsin inhibitor activity in different BBi-mutated lines was measured following the procedure described by Kim et al., Methods in Enzymology; Jez, J., Ed.; Academic Press: Cambridge, MA, USA, 2023; Volume 680, pp. 195-213. Briefly, 20 mg of dry soybean seed powder was placed in a 2 mL Eppendorf centrifuge tube. To this, 1 mL of 10 mM NaOH solution was added and vigorously agitated on a vortex mixer for 10 min at room temperature. The resulting slurry was clarified by centrifugation at 16,000×g for 10 min. Clear supernatant was utilized for measuring KTi activity. Trypsin inhibitor activity was expressed as trypsin units inhibited (TUI) per mg of sample, and results are expressed as the mean±SD from three biological replicates. Chymotrypsin inhibitor activity in soybean seed extracts were measured N-Succinyl-Ala-Ala-Pro-Phe p-nitroanilide (AAPF) as the substrate. Soybean seed extract, which resulted in 40 to 60% chymotrypsin inhibition range, was added to a 1.5 mL Eppendorf tube containing 900 μL of assay buffer (100 mM-Tris-HCL, pH 8.0), 8 μL of α-chymotrypsin (Sigma-Aldrich Company) dissolved in 1 mM HCl solution (0.1 mg/mL), and seed extract (8 to 10 μL) and incubated at 37 °C for 5 min. Following this step, 80 μL of AAPF (1 mg/mL) was added and incubated for an additional 10 min at 37 °C. The assay was stopped by adding 500 μL of 30% acetic acid. Absorbance at 410 nm was measured, and chymotrypsin inhibitor units were calculated as the amount of inhibitor that reduced the absorbance at 410 nm by 0.1 optical density. The results are expressed as the mean±SD from three biological replicates. Trypsin and chymotrypsin inhibitor assay data were visualized and compared using JMP statistical software 16.0 (SAS Institute Inc., Cary, NC, USA). A one way ANOVA was performed, and significant differences (α=0.05) between means were determined using the Tukey-Kramer HSD test.

RNA-Seq Analysis: Total RNA was isolated from developing seeds of the BBi mutant (ZB 168-1) and the wild-type soybean cultivar ‘Maverick’ at three distinct reproductive stages: early (R5), middle (R6), and late (R7). RNA extraction was performed using the TRIzol® Reagent (Thermo Fisher Scientific, Waltham, MA, USA). For each developmental stage, three biological replicates were included. The total RNA was treated with DNase I (Invitrogen, Carlsbad, CA, USA) to remove any contaminating genomic DNA. The RNAseq libraries were prepared by Azenta Life Sciences (Burlington, MA, USA). The process involved the conversion of single-stranded RNA into double-stranded complementary DNA (cDNA), with the addition of sequencing adapters and barcodes, which are subsequently analyzed using next-generation sequencing (NGS). Data analysis was conducted, and the identification of differentially expressed genes (DEGs) was determined.

Confocal Immunofluorescence Microscopy Localization of Bowman-Birk Protease Inhibitor: Soybean seeds soaked in water were cut into several small cubes, using a razor blade. The dissected seed samples were immediately fixed, as previously described in Kunitz et al., J. Gen. Physiol. 1947, 30, 291-310, in a solution containing 2% paraformaldehyde and 2% glutaraldehyde, in 100 mM cacodylate buffer, pH 7.2. After fixation, the samples were dehydrated through the use of a graded ethanol series (80-95-100%) and embedded in paraffin. The paraffin-embedded samples were then processed for confocal immunofluorescence microscopy. Immunohistochemical localization was conducted on paraffin-embedded sections, utilizing antibodies against Bowman-Birk protease inhibitor peptides, raised in rabbits. Five micrometer sections of paraffin-embedded soybean cotyledons were mounted onto X-tra Plus microscope slides (Leica, Richmond, IL, USA), de-waxed in xylene, rehydrated through the use of graded ethanol concentrations, and finally in water. The sections were incubated for 60 min at room temperature, with a 1:200 dilution of the soybean BBi antibody, followed by a 30 min incubation with Alexa Fluor 488 Plus-conjugated goat anti-rabbit secondary antibody (Invitrogen/Thermo Fisher, Waltham, MA, USA), diluted 1:500. The sections were then cover slipped with a mounting medium containing an antifade reagent and observed under a Leica SP8 laser scanning confocal microscope (Leica Microsystems, Buffalo Grove, IL, USA), with a 20×/NA 0.7 objective, using a 495 nm excitation laser line and a 505-550 nm bandpass.

Protein, Oil, and Amino Acid Analyses: The protein and oil content were determined using near-infrared spectroscopy (FOSS North America, Eden Prairie, MN, USA). The amino acid analysis of the wild-type (Maverick) and BBi knockdown line (ZB168-1) was performed at the University of Missouri Agriculture Experiment Station Chemical Laboratories, University of Missouri. The amino acids were separated using a Beckman 6300 Amino Acid Analyzer (Beckman Coulter Life Sciences, Indianapolis, IN, USA), equipped with a high-performance, cation exchange resin column. The results are reported as the mean±standard deviation from three biological replicates.

In Vitro Protein Digestibility Assay: Seed proteins from the wild-type (Maverick) and BBi knockdown line (ZB168-1) were isolated by extracting 30 mg of dry soybean seed powder with 1 mL of 10 mM Tris-HCl, pH 7.5, in a rotary shaker, for 20 min at room temperature. The slurry was centrifuged at 14,000 rpm for 3 min and the resulting supernatant was saved in separate tubes. To this supernatant, 10 μL of trypsin (10 mg/mL) and 10 μL of chymotrypsin (10 mg/mL) stock solution was added and incubated in rotary shaker, maintained at 37 °C. At intervals of 0, 5, 15, 60, and 120 min, 50 μL of protein solution was removed. To this 10 μL of 6×SDS Laemmli sample buffer was added, mixed thoroughly and placed in a boiling water bath for 5 min. The protein samples were resolved on 13.5% SDS-PAGE gels and the separated proteins were visualized by Coomassie Brilliant Blue staining.

### Example: KTI/BBI Double Mutant Promotes Broiler Chicken Growth

The KTI/BBI double mutant soybean line (USDA-HBK-G7.8) and the wild-type soybean line Maverick were evaluated in broiler chickens. In particular, the effects of raw KTI/BBI double mutant soybeans on growth performance and pancreas size in broiler chickens was assessed. A standard commercial soybean meal (SBM) was used for comparison.

Ross 308 male chicks (average 8-day weight: 138.7 g) were weighed, wing-banded, and randomly assigned to dietary treatments in a Completely Randomized Design consisting of three dietary treatment groups, Maverick, USDA-HBK-G7.8, and Commercial SBM. Each treatment group included five replicates, with five chicks allocated to each replicate. Chicks were housed in thermostatically controlled starter batteries with raised-wire flooring in an environmentally controlled room under continuous lighting. Feed and water were provided ad libitum throughout the study. Feed intake was recorded by pen, and final body weights were collected individually at the end of the experiment. Weight gain, feed intake, and gain:feed ratio were calculated. Data were analyzed using ANOVA for a one-way completely randomized design (PROC GLM, SAS).

The results demonstrated that the KTI/BBI double mutant line outperformed Maverick in weight gain, feed intake, and gain:feed ratio (Tables 4-6). Notably, the double mutant supported body weight gain comparable to standard SBM. Additionally, birds fed the KTI/BBI double mutant showed no adverse effects on pancreas size (Tables 7 and 8), indicating suitability for direct feeding. In contrast, birds fed raw Maverick soy exhibited enlarged pancreases.

These findings indicate that raw KTI/BBI double mutant soybeans can serve as a direct protein source in broiler diets, reducing or eliminating the need for energy-intensive processing. Furthermore, the double mutant line has the potential to replace processed SBM without compromising growth performance. Use of KTI/BBI double mutant soybeans as a direct protein source in animal diets would be expected to advantageously lower feed costs for producers and reduce the environmental footprint associated with soybean processing.

TABLE 3
Ingredient composition of experimental diets.

| Ingredients | Diets 1-2 | Diet 3 |
|---|---|---|
| Corn | 57.20 | 66.44 |
| Soy* | 38.00 | 28.76 |
| Limestone | 1.10 | 1.10 |
| Dicalcium Phosphate | 1.94 | 1.94 |
| Salt | 0.21 | 0.21 |
| Sodium Bicarbonate | 0.20 | 0.20 |
| L-Lys HCl | 0.10 | 0.10 |
| DL Met | 0.25 | 0.25 |
| L-Thr | 0.05 | 0.05 |
| Vitamin Mix | 0.20 | 0.20 |
| Mineral Mix | 0.15 | 0.15 |
| Choline Chloride | 0.10 | 0.10 |
| Titanium Dioxide | 0.50 | 0.50 |

*Soy was the ingredient that was tested so it changed for each treatment.

TABLE 4
Growth performance of chicks fed experimental diets. Shown
is weight gain from days 8-19 (g/chick). Means with different
superscripts are significantly different, P < 0.0001.

| | Maverick | USDA-HBK-G7.8 | Commercial SBM |
|---|---|---|---|
| R1 | 345.2 | 543.2 | 531.6 |
| R2 | 303.0 | 501.4 | 536.6 |
| R3 | 334.0 | 538.6 | 524.0 |
| R4 | 343.6 | 583.8 | 548.8 |
| R5 | 316.4 | 498.6 | 552.6 |
| mean | 328.4 ^{c} | 533.1^{a} | 538.7^{a} |
| g/c/d | 27.3 | 44.4 | 44.9 |

TABLE 5
Feed consumption of chicks fed experimental diets.
Shown is feed consumption from days 8-19 (g/chick).

| | Maverick | USDA-HBK-G7.8 | Commercial SBM |
|---|---|---|---|
| R1 | 592.4 | 753.2 | 768.6 |
| R2 | 637.6 | 753.4 | 771.8 |
| R3 | 637.8 | 802.6 | 745.6 |
| R4 | 652.2 | 810.4 | 796.0 |
| R5 | 589.0 | 737.0 | 782.4 |
| mean | 621.8 ^{b} | 771.3 ^{a} | 772.9 ^{a} |
| g/c/d | 51.8 | 64.2 | 64.4 |

TABLE 6
Gain/Feed of chicks fed experimental diets.
Shown is gain/feed from days 8-19 (g/kg).

| | Maverick | USDA-HBK-G7.8 | Commercial SBM |
|---|---|---|---|
| R1 | 583 | 721 | 692 |
| R2 | 475 | 666 | 695 |
| R3 | 524 | 671 | 703 |
| R4 | 527 | 720 | 689 |
| R5 | 537 | 677 | 706 |
| mean | 529 ^{c} | 691 ^{a} | 697 ^{a} |

TABLE 7
Pancreas weight of chicks fed experimental diets.
Shown are pancreas weights (g) at day 19 (g/chick).

| | Maverick | USDA-HBK-G7.8 | Commercial SBM |
|---|---|---|---|
| R1 | 5.01 | 3.25 | 3.26 |
| R2 | 4.57 | 2.84 | 3.04 |
| R3 | 5.85 | 3.03 | 2.98 |
| R4 | 5.23 | 3.35 | 3.25 |
| R5 | 4.17 | 2.98 | 2.97 |
| mean | 4.9 ^{a} | 3.1 ^{b} | 3.1 ^{b} |

TABLE 8
Pancreas weight as % body weight of chicks fed experimental
diets. Shown are pancreas weights (g) as percentage
of body weights (g) at day 19 (g/chick).

| | Maverick | USDA-HBK-G7.8 | Commercial SBM |
|---|---|---|---|
| R1 | 1.04 | 0.47 | 0.48 |
| R2 | 1.03 | 0.44 | 0.45 |
| R3 | 1.23 | 0.44 | 0.45 |
| R4 | 1.08 | 0.46 | 0.47 |
| R5 | 0.91 | 0.46 | 0.43 |
| mean | 1.06 ^{a} | 0.45 ^{c} | 0.45 ^{c} |

### ADDITIONAL NUMBERED EMBODIMENTS

Embodiment 1. A genetically modified soybean seed comprising a loss of function mutation in at least one Bowman-Birk protease inhibitor (BBi) gene, wherein the soybean seed has at least a 2-fold reduction in chymotrypsin inhibitor activity and at least a 2-fold reduction in trypsin inhibitor activity compared to a reference soybean without the BBi mutation.

Embodiment 2. The genetically modified soybean seed of Embodiment 1, wherein the loss of function mutation results in a reduction or elimination of expression of at least one BBi gene compared to a reference soybean seed without the mutation.

Embodiment 3. The genetically modified soybean seed of Embodiment 1 or 2, wherein the at least one BBi gene is Glyma.16g208900 or Glyma.14G117700, or both.

Embodiment 4. The genetically modified soybean seed of Embodiment 3, wherein the at least one BBI gene comprises one or more of Glyma.09G158500, Glyma.09G158600, and Glyma.09G158700.

Embodiment 5. The genetically modified soybean seed of any one of Embodiments 1 to 4, wherein the seed contains similar amounts of Kunitz protease inhibitor (KTi) protein compared to a reference soybean seed without the mutation.

Embodiment 6. The genetically modified soybean seed of any one of Embodiments 1 to 4, comprising a loss of function mutation in one or more Kunitz protease inhibitor (KTi) isoforms.

Embodiment 7. The genetically modified soybean seed of Embodiment 6, comprising a loss of function mutation in a KTi1, KTi2, or KTi3 isoform.

Embodiment 8. The genetically modified soybean seed of Embodiment 6, comprising a loss of function mutation in both KTi1 and KTi3 isoforms.

Embodiment 9. The genetically modified soybean seed of any one of Embodiments 1 to 8, wherein the loss of function mutation in the at least one BBi gene is a deletion, insertion, or frameshift mutation resulting in one or more of reduced BBi gene expression, an enzymatically inactive BBi protein or a BBi protein having reduced enzymatic activity compared to a reference BBi protein without the mutation.

Embodiment 10. The genetically modified soybean seed of Embodiments 1 to 9, wherein the species of the soybean seed is Glycine max (L.) Merrill.

Embodiment 11. A genetically modified soybean or soybean meal comprising a loss of function mutation in at least one Bowman-Birk protease inhibitor (BBi) gene, wherein the soybean or soybean meal has at least a 2-fold reduction in chymotrypsin inhibitor activity and at least a 2-fold reduction in trypsin inhibitor activity compared to a reference soybean or soybean meal without the BBi mutation.

Embodiment 12. The genetically modified soybean or soybean meal of Embodiment 11, wherein the at least one BBi gene is Glyma.16g208900 or Glyma.14G117700, or both.

Embodiment 13. The genetically modified soybean or soybean meal of Embodiment 11 or 12, wherein the soybean or soybean meal has not been subjected to heat treatment.

Embodiment 14. The genetically modified soybean or soybean meal of any one of Embodiments 11 to 13, wherein trypsin or chymotrypsin inhibitor activity measured in the soybean or soybean meal is less than 50%, less than 20% or less than 10% of the activity of a reference soybean or meal not carrying the BBi mutation.

Embodiment 15. The genetically modified soybean or soybean meal of any one of Embodiments 11 to 13, wherein the trypsin inhibitor activity is less than 2.5 milligrams/gram or less than 0.25% by weight.

Embodiment 16. The genetically modified soybean or soybean meal of any one of Embodiments 11 to 15, wherein the species of the soybean is Glycine max (L.) Merrill.

Embodiment 17. The genetically modified soybean or soybean meal of any one of Embodiments 11 to 16, wherein the soybean is a Maverick cultivar.

Embodiment 18. An animal feed mixture comprising the genetically modified soybean or soybean meal of any one of Embodiments 11 to 17.

Embodiment 19. A method for reducing trypsin and chymotrypsin inhibitor activity in soybean or soybean seed, or in a meal or flour produced from the soybean or soybean seed, the method comprising producing a transgenic soybean plant comprising one or more loss of function mutations in at least one Bowman-Birk protease inhibitor (BBi) gene.

Embodiment 20. The method of Embodiment 19, wherein the at least one BBi gene comprises one or both of Glyma.16g208900 and Glyma.14G117700.

Embodiment 21. The method of Embodiment 19 or 20, wherein trypsin or chymotrypsin inhibitor activity of the soybean, seed, or in a meal or flour produced from the soybean seed, is less than 50% or less than 20% or less than 10% of the activity of a reference soybean not carrying the BBi mutation, or in a meal or flour produced from the reference soybean seed.

Embodiment 22. The method of Embodiment 19 or 20, wherein the trypsin inhibitor activity of the soybean or soybean seed, or in a meal or flour produced from the soybean seed, is less than 2.5 milligrams/gram or less than 0.25%.

Embodiment 23. The method of any one of Embodiments 19 to 22, wherein the method does not comprise subjecting the soybean or seed, or a meal or flour produced from the soybean seed, to a heat treatment.

Embodiment 24. The method of Embodiment 23, wherein the heat treatment comprises one or more of roasting, extrusion, and/or steam treatment.

Embodiment 25. The method of Embodiment 23 or 24, wherein the heat treatment comprises exposure to a temperature of between 100-110 °C for a period of time ranging from minutes to hours.

Embodiment 26. The method of any one of Embodiments 23 to 25, further comprising exposure to a temperature up to 150 °C.

Embodiment 27. The method of any one of Embodiments 19 to 22, wherein the method comprises subjecting the soybean, seed, or a meal or flour produced from the soybean seed, to a modified heat treatment wherein the heat treatment comprises one or more of exposure to a lower temperature and/or exposure for a shorter period of time compared to a heat treatment required to produce the same trypsin or chymotrypsin inhibitor activity in a soybean, seed, or a meal or flour produced from the soybean seed not carrying the BBi mutation.

Embodiment 28. The method of any one of Embodiments 19-23 or 27, wherein the method does not comprise mutating a Kunitz protease inhibitor (KTi) gene.

Embodiment 29. The method of any one of Embodiments 19-23 or 27, wherein the soybean comprises a loss of function mutation in one or more Kunitz protease inhibitor (KTi) gene isoforms, optionally wherein the KTi isoform is a KTi1, KTi2, or KTi3 isoform.

Embodiment 30. The method of any one of Embodiments 19-23 or 27-29, wherein the one or more loss of function mutations in the at least one BBi gene is introduced by a method comprising Agrobacterium-mediated transformation of an expression cassette encoding at least one guide RNA targeting the at least one BBi gene.

Embodiment 31. The method of any one of Embodiments 19-23 or 27-30, wherein the one or more loss of function mutations is introduced using a CRISPR/Cas9 system.

Embodiment 32. The method of any one of Embodiments 19-23 or 27-31, wherein the species of the transgenic soybean plant is Glycine max (L.) Merrill.

Embodiment 33. The method of any one of Embodiments 19-23 or 27-32, wherein the transgenic soybean plant is a Maverick cultivar.

While the invention herein disclosed has been described by means of specific embodiments and applications thereof, modifications and variations could be made thereto by those skilled in the art without departing from the scope of the invention set forth in the claims.

It will be appreciated that the present invention is set forth in various levels of detail in this application. In certain instances, details not necessary for one of ordinary skill in the art to understand the invention, or that render other details difficult to perceive may have been omitted. Unless defined otherwise, technical terms used herein are to be understood as commonly understood by one of ordinary skill in the art to which the disclosure belongs.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” means that the value may vary by +/−10%.

In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “cell line” refers to a genetically homogenous population of cells that can be cultured or propagated under in vitro conditions. In this context, a “stable” genetically modified cell line is one where the genetic modifications are heritable.

The term “tissue culture” refers to a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollens, flowers, seeds, leaves, stems, roots, root tips, anthers, pistils, meristematic cells, axillary buds, ovaries, seed coats, endosperms, hypocotyls, cotyledons and the like. Means for preparing and maintaining plant tissue culture are well known in the art.

What is claimed is:

1. A genetically modified soybean seed comprising a loss of function mutation in at least one Bowman-Birk protease inhibitor (BBi) gene, wherein the soybean seed has at least a 2-fold reduction in chymotrypsin inhibitor activity and at least a 2-fold reduction in trypsin inhibitor activity compared to a reference soybean seed without the BBi mutation.

2. The genetically modified soybean seed of claim 1, wherein the loss of function mutation results in a reduction or elimination of expression of at least one BBi gene compared to a reference soybean seed without the mutation.

3. The genetically modified soybean seed of claim 1, wherein the at least one BBi gene is Glyma.16g208900 or Glyma.14G117700, or both.

4. The genetically modified soybean seed of claim 3, wherein the at least one BBI gene comprises one or more of Glyma.09G158500, Glyma.09G158600, and Glyma.09G158700.

5. The genetically modified soybean seed of claim 1, wherein the seed is designated USDA-168-1, USDA-168-4, USDA-168-5, USDA-Cas9-2.8, USDA-Cas9-7.2 or USDA-Cas9-8.7.

6. The genetically modified soybean seed of claim 1, comprising a loss of function mutation in one or more Kunitz protease inhibitor (KTi) isoforms.

7. The genetically modified soybean seed of claim 6, comprising a loss of function mutation in a KTi1, KTi2, or KTi3 isoform.

8. The genetically modified soybean seed of claim 6, comprising a loss of function mutation in both KTi1 and KTi3 isoforms.

9. The genetically modified soybean seed of claim 8, wherein the seed is designated USDA-HBK-G7.8.

10. A soybean plant, a part thereof, or a cell thereof produced by growing the genetically modified soybean seed of claim 1.

11. The soybean plant or the part thereof of claim 10, wherein the part is selected from the group consisting of a leaf, a flower, a soybean, a stalk, a root, a rootstock, a scion, a peduncle, a stamen, a pistil, and a meristem.

12. A progeny of the genetically modified soybean seed of claim 1 produced from a cross of a plant or plant part of the genetically modified soybean seed with a different Glycine max (L.) Merrill plant.

13. A line of regenerable cells produced from the soybean plant or the part thereof of claim 10, wherein the soybean plant regenerated from a tissue culture of the cells has all of the physiological and morphological characteristics of the genetically modified soybean seed of claim 1.

14. The genetically modified soybean seed of claim 1, wherein the loss of function mutation in the at least one BBi gene is a deletion, insertion, or frameshift mutation resulting in one or more of reduced BBi gene expression, an enzymatically inactive BBi protein or a BBi protein having reduced enzymatic activity compared to a reference BBi protein without the mutation.

15. A genetically modified soybean or soybean meal produced from the genetically modified soybean seed of claim 1.

16. The genetically modified soybean or soybean meal of claim 15, wherein the soybean or soybean meal has not been subjected to heat treatment.

17. The genetically modified soybean or soybean meal of claim 15, wherein trypsin or chymotrypsin inhibitor activity measured in the soybean or soybean meal is less than 50% of the activity of a reference soybean or meal, optionally wherein the trypsin inhibitor activity is less than 2.5 milligrams/gram or less than 0.25% by weight.

18. An animal feed mixture comprising the genetically modified soybean or soybean meal of claim 15.
